(19) United States
(12) Patent Application Publication
(10) Pub. No.: US 2024/0215905 A1
(43) Pub. Date: Jul. 4, 2024

(54) WOUND DRESSING

(71) Applicant: 5D HEALTH PROTECTION GROUP LTD, Gwynedd (GB)

(72) Inventors: Neville J. Freeman, Tarvin, Cheshire (GB); Stephen Law, Nuneaton, Warwockshire (GB); Steven L. Percival, Chester, Cheshire (GB); Marcus J. Swann, Lymm, Cheshire (GB)

(21) Appl. No.: 18/572,513

(22) PCT Filed: Jun. 30, 2022
(86) PCT No.: PCT/GB22/51684

§ 371 Date: Dec. 20, 2023

(30) Foreign Application Priority Data
Jun. 30, 2021 (GB) 2109440.4

(51) Int. Cl. A61B 5/00; A61B 5/1477; A61F 13/00

(52) U.S. Cl. CPC A61B 5/445; A61B 5/1477; A61B 5/6833; A61F 13/00051; A61B 2562/028; A61B 2562/0285; A61F 2013/00442

(57) ABSTRACT

The present invention relates to a wound dressing comprising an electrochemical sensor configured to detect the level of an electroactive species within a wound environment during use. The present invention also relates to a method of detecting the level of an electroactive species within a wound environment by applying a wound dressing according to the present invention onto a wound and operating an electrochemical sensor to detect an electroactive species within the wound environment.

### FIELD OF THE INVENTION

The present invention relates to a wound dressing comprising an electrochemical sensor configured to detect the level of an electroactive species within a wound environment during use. The present invention also relates to a method of detecting the level of an electroactive species within a wound environment by applying a wound dressing according to the present invention onto a wound and operating an electrochemical sensor to detect an electroactive species within the wound environment.

### BACKGROUND OF THE INVENTION

Wound care remains a major cost to developed healthcare economies around the globe. There is an immediate and pressing need to improve the efficiency of wound care management. The changing of a wound dressing too early risks disturbing the wound bed, impeding the healing process and potentially introducing infection. Changing the dressing too late risks the wound dressing becoming infested by microorganisms and, in the case of an ‘at risk’ of infection or infected wound, potentially supporting the microorganisms responsible for impeding the healing process.

It is common clinical practice to utilise a dressing that incorporates an anti-microbial active agent (e.g. silver ions) to treat wounds, especially infected wounds. If too little anti-microbial is used, the dressing will fail to control the advance of unwanted microbes, whilst too much risks introducing toxicity. It is therefore desirable to provide an optimum and therapeutic concentration of anti-microbial to improve healing. Another problem associated with the use of dressings containing anti-microbial agents, such as silver ions, is that whilst the dressing itself often contains an excess of the anti-microbial agent this does not reflect the actual amount of anti-microbial material that is biologically available within the wound environment. The consequence of this is that more anti-microbial material tends to be used than is required to be effective. This can of course impact upon the downstream waste produced with potentially detrimental environmental consequences on disposal, especially where metal ion anti-microbial agents are utilised. Optimal and effective anti-microbial delivery levels are currently not very well understood and it is therefore desirable for clinical professionals to be able to determine the analyte levels (e.g. anti-microbial levels) within a wound environment to better inform clinical decisions regarding the management of such wound environments and the associated dressings.

Currently known methods of monitoring analytes in wound dressings typically include the use of electrochemical sensors which utilise relatively large “macro” electrodes as the working (or sensing) electrode, printed onto a planar film support substrate. Typical macro electrodes are described in Tables 1 and 2 below. Macro electrodes have been used previously due to their relative ease of manufacture and integration into wound dressing structures. The present inventors have however established that such wound dressings suffer from drawbacks in clinical settings. One major problem associated with the use of macro electrodes relates to the difficulty in incorporating such electrodes into a wound dressing without compromising the dressing's performance characteristics. For example, when fabricated with a macro electrode on a supporting film, the fluid flow properties and ability of a dressing to absorb wound fluids (e.g. wound exudate) from the wound is significantly impacted. This occurs as macro electrodes and their associated components assume a large footprint within a wound dressing structure. This, when the dressing is applied to an open wound, occludes and reduces the flow of wound fluid from the open wound into the wound dressing and likewise occludes the flow of species within the dressing both to the sensor and to the wound.

In addition to the diminished fluid flow and absorbency properties, the present inventors have also identified that wound dressings that contain macro electrodes also possess relatively poor electroactive analyte detection sensitivity in a wound environment, especially when used to detect low analyte concentration levels which are typical of a wound environment. This is particularly the case for planar electrodes. This is because the act of polarising a working macro electrode, which is necessary for measurement and detection, causes a double layer charging current to be passed through the solution between the working electrode and counter electrode. Where the electroactive analyte concentration is low, this charging current often represents a significant portion of the detection signal and therefore limits the signal to noise ratio of the analyte measurement. Without being bound by theory, this is partly because macro electrodes suffer from mass transport effects whereby analyte species (e.g. electroactive species) within wound fluid (e.g. wound exudate) present in the wound environment are rapidly consumed by the electrode and need to diffuse over significant distances to reach the surface of the working electrode for detection. An additional problem is that wound dressings are generally designed to keep the wound bed moist, but to absorb excess fluid, so that the volume of fluid in the vicinity of the sensor electrodes may be low and the viscosity high which also hinders transport of the analyte to the sensor surface. A further consequence of this is that any stirring induced by movement in the dressing may cause large fluctuations in the measured signal.

It is therefore desirable to obviate or mitigate one or more of the above problems with the prior art whilst still achieving desirable analyte detection capability and ideally without significantly diminishing the performance characteristics of the dressing.

### SUMMARY OF THE INVENTION

The present inventors have developed a wound dressing containing an electrochemical sensor which is eminently suitable for the detection of electroactive species, especially electroactive anti-microbial and anti-biofilm agents, within a wound environment during use.

In a first aspect there is provided a wound dressing comprising an electrochemical sensor configured to detect the level of an electroactive species within a wound environment during use, the electrochemical sensor comprising a working electrode, wherein the working electrode is a micro electrode or nano electrode.

In a second aspect there is provided a method of detecting the level of an electroactive species within a wound environment comprising applying a wound dressing according to the first aspect of the present invention, or any embodiment thereof, to a wound and operating the electrochemical sensor to detect an electroactive species within a wound environment.

In a third aspect there is provided a use of a wound dressing according the first aspect of the present invention, or any embodiment thereof, in detecting the level of an electroactive species within a wound environment.

In a fourth aspect there is provided a use of a wound dressing in the method of the second aspect of the present invention or any embodiment thereof.

Also provided is an electroactive anti-microbial agent for use in treating a wound in a patient (e.g. a wound infected by microbes, such as bacteria), the electroactive anti-microbial agent being present within the wound dressing according to the first aspect, wherein the method comprises applying the wound dressing to the wound and operating the electrochemical sensor to detect the electroactive anti-microbial species within the wound environment.

Also provided is a use of an electroactive anti-microbial agent in a method of manufacturing a therapy for treating a wound in a patient (e.g. a wound infected by microbes, such as bacteria), the electroactive anti-microbial agent being present within the wound dressing according to the first aspect, wherein the method comprises applying the wound dressing to the wound and operating the electrochemical sensor to detect the electroactive anti-microbial agent within the wound environment.

Also provided is a method of treating a wound in a patient (e.g. a wound infected by microbes, such as bacteria), the method comprising applying the wound dressing according to the first aspect to the wound and operating the electrochemical sensor to detect the electroactive species within the wound environment, e.g. wherein the electroactive species is an electroactive anti-microbial or anti-biofilm agent.

The wound dressings of the present invention advantageously incorporate working electrodes that are micro electrodes or nano electrodes. Such electrodes are able to obviate or mitigate problems identified with conventional solutions which adopt macro electrodes, described herein above. The micro or nano electrodes of the present invention allow for improved fluid flow and absorbency within the dressing compared to dressings which use macro electrode-based wound dressing systems thus overcoming the mass transport effects experienced by macro electrodes. Whilst the reduced footprint of the micro electrodes and nano electrodes play an important role in achieving this advantage, it has also been found that micro electrodes and nano electrodes can be more easily integrated into the suitable wound dressing materials without detrimental effects as described in more detail herein. As described above, the signal to noise ratio of conventional macro electrodes, can be low, thus making it difficult or impossible to detect low concentrations of analytes in the wound environment. When using micro and nano electrodes in a wound dressing according to the present invention, this “noise” level is advantageously relatively low which, as a result, leads to the signal to noise ratio increasing and thus an improvement in the detection sensitivity relative to macro electrodes. This is particularly apparent when utilising working electrodes which are wires, as described herein, since the electroactive analyte species present in a wound environment is able to diffuse to the surface of the working electrode for detection from an area radially surrounding the working electrode. This is not the case with planar macro electrodes which generally allow for a one-dimensional diffusion of the electroactive species within the wound environment to the surface of the working electrode, which results in rapid depletion of the electroactive analyte close to the electrode surface and decay of the detection signal in use.

As referred to herein, the term “wound dressing” includes dressings suitable for application to a site on a human or animal subject's body for the purpose of protecting the site from its external environment and/or aiding a healing process at the site. The site may include a wound, skin or nail of the subject. The site may also include areas on and surrounding areas of catheters and catheter insertion points, orthopedic devices and areas surrounding stomas. Typically, the wound dressing is suitable for wounds formed on the skin, nail or tissue of the human or animal subject. The wound dressings are suitable for any type of wound, in particular, surface wounds or cavity wounds. In some instances, the wound may include skin abrasions, lacerations, punctures, surgical incisions, avulsions, burns, ulcers and other such like wounds.

Wound dressings of the present invention may perform numerous functions including, but not necessarily limited to, absorbing wound exudate, promotion of wound granulation and healing, administration of active agents and/or protecting the wound from its external environment. Wound dressings according to the present invention may typically include a dressing layer (or absorbent layer) which may be woven or non-woven and can be secured to the site of a wound using a suitable adhesive (e.g. adhesive tape or adhesive layer etc.). In some embodiments, the wound dressing is an island dressing comprising a backing layer, an absorbent layer, and a wound contact layer that contains an adhesive. An electrochemical sensor, as described herein, may be disposed on or in any layer forming part of the wound dressing, provided it is configured to detect the level of an electroactive species within a wound environment during use.

It will be appreciated that the term “wound environment” refers to any part of the wound dressing and the site of the wound, located on a human or animal subject, on which the dressing is applied during use. It will be appreciated that the wound environment includes any area of the wound dressing and wound in which an electroactive species can reside and be detected during the wound healing process. For the avoidance of doubt, wound environment includes any part of the wound dressing structure and material (e.g. within the fibres or filaments forming part of an absorbent layer within the wound dressing), the area within the wound itself and its surrounding area (e.g. surrounding skin), and/or any wound fluid present within the wound, its surrounding area or the wound dressing during use.

In suitable embodiments, the “micro electrode” according to the present invention has a minimum dimension of greater than or equal to 1 μm and less than or equal to 500 μm. The minimum dimension may be greater than or equal to 1 μm and less than or equal to 150 μm, e.g. greater than or equal to 1 μm and less than or equal to 100 μm, such as greater than or equal to 1 μm and less than or equal to 50 μm, or greater than or equal to 1 μm and less than or equal to 25 μm, or greater than or equal to 1 μm and less than or equal to 10 μm, or greater than or equal to 1 μm and less than or equal to 5 μm. For example, where the micro electrode is a micro-wire, it will be understood that the minimum dimension measurement of the micro-wire corresponds to the diameter of the wire. Exemplary micro-wire electrodes suitable for use in wound dressings of the present invention are described in Tables 1 and 2 below (referred to as “Pt Micro-wire (100 μm)”, “Pt Micro-wire (25 μm)”, “Pt Micro-wire (5 μm)” and “Pt Micro-wire (1 μm)”). The “μm” measurement values found within the brackets of the reference name used for each of the micro electrodes within Tables 1 and 2 correspond to the diameter measurement for each of the respective micro-wires. In such embodiments, for micro-wire electrodes, the surface area of the working electrode that is exposed to the wound environment during use may be from about 6 mm² to about 8 mm², from about 2 mm² to about 3 mm², from about 1 mm² to about 2 mm², from about 0.5 mm² to about 1 mm², from about 0.1 mm² to about 0.2 mm² or from about 0.01 mm² to about 0.05 mm².

In embodiments, the “micro electrode” according to the present invention has a maximum dimension of greater than or equal to 1 mm. For example, the “micro electrode” according to the present invention may have a maximum dimension of from about 1 mm to about 10 cm or from about 1 mm to about 20 cm.

In suitable embodiments, the “nano electrode” has a minimum dimension of less than 1000 nm (i.e. less than 1 μm). An exemplary nano electrode suitable for wound dressings according to the present invention, referred to as a “Nanoband electrode”, is described in Tables 1 and 2 below. The Nanoband electrode possesses an electrode structure analogous to the electrode structure depicted in FIG. 3. Here, a planar laminate structure containing a thin, electrically conductive working electrode layer (labelled as 16 in FIG. 3), an insulating layer (labelled as 22 in FIG. 3) and substrate layer (labelled as 20 in FIG. 3) is used to create “etched out” apertures (labelled as 24 in FIG. 3). These apertures provide exposed edge sensing surfaces formed of the working electrode material (labelled as 18 in FIG. 3). It will be appreciated that, when such nano electrodes are used as part of the wound dressing according to the present invention, the minimum dimension measurement corresponds to the thickness (or the height) of the exposed edge forming the sensing surface. This thickness measurement is depicted as “D” in FIG. 3. The exposed edge may have a thickness of from about 5 nm to 200 nm, from about 25 nm to about 200 nm, from about 50 nm to about 200 nm, from about 100 nm to about 200 nm, from about 150 nm to about 200 nm, from about 5 nm to about 150 nm, from about 5 nm to about 100 nm, from about 5 nm to about 50 nm or from about 5 nm to about 25 nm. Preferably, the exposed edge has a thickness of from about 25 nm to about 75 nm or from about 35 nm to about 65 nm or from about 45 nm to about 55 nm. In embodiments, the exposed edges have a thickness of about 50 nm. In such embodiments, the nano electrode has a surface area of the working electrode that is exposed to the wound environment during use may be from about 0.001 mm² to about 0.01 mm² or from about 0.02 mm² to about 0.04 mm².

It will be appreciated that the wound dressings according to the present invention include an electrochemical sensor configured to detect the electroactive species. Detection may be performed via any suitable electrochemical technique. In embodiments, the electrochemical sensor is capable of detecting the electroactive species by anodic stripping voltammetry, amperometry, potentiometry or electrochemical impedance analysis (e.g. electrochemical impedance spectroscopy). Preferably, in some embodiments, the electrochemical sensor is capable of detecting the electroactive species by anodic stripping voltammetry. It will be appreciated that for anodic stripping voltammetry of an electroactive species (e.g. metal ions) it is required that the electroactive species is accumulated on the surface of the working electrode in their reduced state and then re-solubilised once oxidized. In other embodiments, the electrochemical sensor is capable of detecting the electroactive species by impedance analysis. In some embodiments, the electrochemical sensor detects the electroactive species as a result of deposition or breakdown of a layer or coating on the surface of the working electrode. For example the working electrode could be coated with a polymer which was broken down by enzymes released into the wound environment either by the wound itself or by microbes within and outside the dressing or wound bed. In further embodiments, the electrochemical sensor is capable of detecting an electroactive species that is either generated by or consumed by a biofilm or microbial bioburden within the wound environment. For example, the electrochemical sensor is capable of detecting an electroactive metabolite produced by the biofilm.

The electrochemical sensor may be capable of detecting an electroactive species within the wound environment at concentration levels from less than about 10 parts per million (ppm) of the electroactive species. In some embodiments, the electrochemical sensor detects an electroactive species within the wound environment at concentration levels from less than about 5 ppm of the electroactive species. In other embodiments, the electrochemical sensor detects an electroactive species within the wound environment at concentration levels of at least 10 ppb of the electroactive species.

In embodiments, the electrochemical sensor may be capable of generating an output signal representative of the level of the electroactive species detected.

As recited herein, the term “electroactive species” is intended to include analytes that are capable of being oxidised or reduced at the surface of the working electrode according to the present invention, and is therefore able to be detected using suitable electrochemical techniques, such as those described further below. The term is also intended to include analytes that may associate or self-assemble at the working electrode surface during use but without necessarily contacting the electrode surface. Such species are also capable of being detected at the working electrode using suitable electrochemical techniques, such as those described herein. In some instances, the electroactive species may be capable of significantly changing the electrical double layer at the surface of an electrode in response to an applied potential without undergoing oxidation or reduction themselves.

It will be appreciated that the electroactive species may be an active agent suitable for administration into a wound environment. Typically, the active agent is a therapeutic agent suitable for maintaining or improving the status of a wound when administered to the wound environment during the wound healing process. It will be appreciated that such therapeutic agents may cause therapeutic effects when administered to the wound environment in order to maintain or improve the status of the wound and enhance the wound healing process. Examples of suitable therapeutic agents include an anti-microbial agent, anti-biofilm agent, anti-inflammatory agent, anti-biotic agent, anti-viral and/or anti-fungal agent. Additionally, or alternatively, the electroactive species may be a marker (e.g. biological marker including inflammatory agents) that provides an indication of the wound status during the wound healing process. By way of example, the electroactive species may be a diagnostic marker (e.g. metabolite) for a microbe present in the wound environment. It will be understood that the electroactive species may be an active agent (e.g. anti-microbial agent or anti-biofilm agent, etc.) that is administered into the wound environment during use. In some embodiments, the electroactive species is an active agent as described herein which may form any part of the wound dressing during its manufacture and is released from the wound dressing into the surrounding wound environment during use.

In embodiments, the electroactive species is a metal species or a non-metal species.

In some embodiments, an active agent, which is independent of and different to the electroactive species being detected in the wound environment, may be separately administered or present in the wound environment during use of the wound dressing of the present invention. In such embodiments, the active agent may be any one of the active agents as described herein. For example, the active agent may be a therapeutic agent such as an anti-microbial agent, anti-biofilm agent, anti-inflammatory agent, anti-biotic agent, anti-viral and/or anti-fungal agent as described herein. Where the active agent is different to the electroactive species being detected in the wound environment, the active agent may be provided as a component of the wound dressing. It may form any part of the wound dressing and may be released from the wound dressing into the surrounding wound environment during use.

In embodiments, the electroactive species is an active agent which is a therapeutic agent. For example, the electroactive species may be an anti-microbial agent, anti-biofilm agent, anti-inflammatory agent, anti-biotic agent, anti-viral and/or anti-fungal agent. In some embodiments, the electroactive species is an anti-microbial agent and/or anti-biofilm agent, e.g. anti-microbial.

The anti-microbial agent and/or anti-biofilm agent may be a metal selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr. In embodiments, the anti-microbial agent and/or anti-biofilm agent is Ag.

The anti-microbial agent and/or anti-biofilm agent may be a metal ion selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr ions. In embodiments, the anti-microbial agent and/or anti-biofilm agent is a Ag metal ion.

The anti-microbial agent and/or anti-biofilm agent may be a metal nanoparticle formed from a metal selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr. In embodiments, the anti-microbial agent and/or anti-biofilm agent is a metal nanoparticle formed from Ag.

The anti-microbial agent and/or anti-biofilm agent may be a non-metal species. For example, in some embodiments, the anti-microbial agent and/or anti-biofilm agent is a reactive oxygen species. Typically, the reactive oxygen species may include superoxide radical anions (O₂·—), peroxides (such as hydrogen peroxide, H₂O₂), hydroxyl radicals (OH·), hypochlorous acid (HOCl), hypobromous acid (HOBr), hypoiodous acid (HOI), peroxyl radicals (ROO·), alkoxyl radicals (RO·), semiquinone radicals (SQ·-) and carbonate radicals (CO₃·—), organic hydroperoxides (ROOH), organic peroxide acids (RO₃H), ozone (O₃), singlet oxygen (¹O₂) or any precursor compound suitable for producing any one of the aforementioned reactive oxygen species within a wound environment. In some embodiments, the anti-microbial agent and/or anti-biofilm is a halogen, typically, chlorine, bromine or iodine, or a halide such as chloride, bromide or iodide. In other embodiments, the anti-microbial agent and/or anti-biofilm is oxygen, perchlorous acid, hydrochlorous acid or a hyperchlorite ion. In further embodiments, the anti-microbial agent and/or anti-biofilm agent is reactive nitrogen species, for example, peroxynitrite (ONOO·).

Other examples of electroactive species according to the present invention include peptides, peracetic acid, organic acids such as benzoic acid, citric acid, lactic, malic or tannic acid, or organic salts such as benzalkonium chloride and quaternary ammonium compounds.

In embodiments, the electroactive species is a surfactant (cationic, anionic and neutral surfactants), typically, a poloxamer surfactant. In some embodiments, the electroactive species is a chelating agent. Typical chelating agents include EDTA (e.g. di-sodium EDTA, tri-sodium EDTA, tetra-sodium EDTA), DPTA, TTHA, EGTA, citrates, and polyphosphates, or a combination of two or more thereof.

In embodiments, the electroactive species is a suitable energy carrying molecule, for example, adenosine triphosphate (ATP) or a derivative thereof.

Where the electroactive species according to the present invention is a peptide and/or an anti-biotic agent, the anti-biotic agent may be selected from the group consisting of aminoclycosides (e.g. gentamicin, neomycin or streptomycin), ansamycins, penicillins (e.g. natural, aminopenicillins and antipseudomonal), amoxicillin, ampicillin, pseudomonic acids, cephalosporins (third, fourth and fifth generation), cephamycins (e.g. ceftiofur, cefadroxil, cefuroxime), spectinomycin, glycopeptides (e.g. avoparcin, vancomycin), macrolides and ketolides (e.g. erythromycin, tilmicosin, tylosin), polymyxins, quinolones (e.g. fluoroquinolones sarafloxacin, enrofloxaxin), streptogramins (e.g. virginiamycin), quinupristin-dalfopristin, carbapenems, lipopeptides, oxazolidinones, cycloserine, ethambutol, ethionamide, isoniazid, para-aminosalicyclic acid, pyrazinamide, sulphonamides (e.g. sulfadimethoxine, sulfamethazine, sulfisoxazole), tetracyclines (e.g. chlortetracycline, oxytetracycline, tetracycline), dofazimine, monobactams, sulfones, polypeptides (e.g. bacitracin), lincosamides (e.g. lincomycin), glycylcyclins, lipopeptides, oxazolidinones, phosphoric acid derivatives, and riminofenazines, miltefosine, polyhexamethylene biguanide (PHMB), octenidine, chlorhexidine or a combination of two or more thereof.

Anti-fungal agents according to the present invention may be selected from the group consisting of azole anti-fungal agents (e.g. imidazole class, triazole class, and thiazole class), echinocandins, polyenes, terbinafine, amphotericin B, candicidin, filipin, hamycin, natamycin, nystatin, and rimocidin, or any combination of two or more thereof. Examples of azole anti-fungal agents according to the present invention include fluconazole clotrimazole, econazole, miconazole and ketoconazole. Other anti-fungal agents may also include aurones, crystal violet, ciclopirox, flucytosine (5-fluorocytosine), griseofulvin, haloprogin, tolnaftate, undecylenic acid, orotomide,

It will be appreciated that the surface area of the working electrode that is exposed to the wound environment (i.e. the exposed surface area of the working electrode) must be large enough to allow the detection of a given electroactive species that is present in the wound environment during use. The exact area of the exposed surface area of the working electrode may be varied depending on the size of the wound, the dressing and the nature of the electroactive species being detected. Similarly, the footprint of the working electrode must be sufficient to probe a representative area of the wound environment.

It has been established that the wound dressing of the present invention includes a working electrode (forming part of an electrochemical sensor) which is unexpectedly and advantageously useful in detecting the level of an electroactive species within a wound environment over an extended area. The wound dressings of the present invention may be utilised to detect the level of an electroactive species within wound environments of varying sizes but without compromising the detection sensitivity of the electrochemical sensor forming part of the wound dressing. It follows that the working electrodes forming part of the wound dressings of the present invention can have a footprint area probed that can be varied depending on the size requirements of the wound environment but whilst maintaining an excellent level of electroactive species detection performance. The footprint area probed of the working electrode may be from about 0.1 mm² to about 1000 mm². In some embodiments, the footprint area probed of the working electrode is from about 5 mm² to about 1000 mm², from about 100 mm² to about 1000 mm², from about 250 mm² to about 1000 mm², from about 500 mm² to about 1000 mm², from about 750 mm² to about 1000 mm², from about 0.1 mm² to about 750 mm², from about 0.1 mm² to about 500 mm², from about 0.1 mm² to about 250 mm² or from about 5 mm² to about 100 mm². In some embodiments, the footprint area probed of the working electrode is from about 1 mm² to about 50 mm², from about 5 mm² to about 50 mm², from about 10 mm² to about 50 mm², from about 20 mm² to about 50 mm², from about 30 mm² to about 50 mm², from about 40 mm² to about 50 mm², from about 1 mm² to about 40 mm², from about 1 mm² to about 30 mm², from about 1 mm² to about 20 mm², from about 1 mm² to about 10 mm² or from about 1 mm² to about 5 mm². Preferably, the footprint area probed of the working electrode is from about 1 mm² to about 100 mm² or from about 1 mm² to about 50 mm².

The surface area of the working electrode that is exposed to the wound environment during use is typically from about 0.0005 mm² to about 10 mm². In some embodiments, the surface area of the working electrode that is exposed to the wound environment during use is from about 0.0005 mm² to about 8 mm², from about 0.0005 mm² to about 6 mm², from about 0.0005 mm² to about 4 mm² or from about 0.0005 mm² to about 2 mm². In embodiments, the surface area of the working electrode that is exposed to the wound environment during use is from about 0.001 mm² to about 8 mm² or from about 0.001 mm² to about 3 mm².

When the working electrode is a micro electrode, typically a wire, the surface area of the working electrode that is exposed to the wound environment during use may be from about 0.01 mm² to about 8 mm², from about 0.01 mm² to about 3 mm². In embodiments, the working electrode is a micro electrode, typically a wire, and the surface area of the working electrode that is exposed to the wound environment during use is from about 6 mm² to about 8 mm², from about 2 mm² to about 3 mm², from about 1 mm² to about 2 mm², from about 0.5 mm² to about 1 mm², from about 0.1 mm² to about 0.2 mm² or from about 0.01 mm² to about 0.05 mm².

When the working electrode is a nano electrode the surface area of the working electrode that is exposed to the wound environment during use may be from about 0.001 mm² to about 0.05 mm². In embodiments, the working electrode is a nano electrode and the surface area of the working electrode that is exposed to the wound environment during use is from about 0.001 mm² to about 0.01 mm² or from about 0.02 mm² to about 0.04 mm².

The solution volume probed (as described below) per exposed surface area of the working electrode (also described below) also referred to herein as “(solution volume probed)/(exposed surface area of the working electrode)” provides an indication of the volume of the wound environment which is probed by the working electrode per surface area of the working electrode. The (solution volume probed)/(exposed surface area of the working electrode) for a working electrode according to the present invention is from about 0.5 mm to about 300 mm. In some embodiments, the (solution volume probed)/(exposed surface area of the working electrode) for a working electrode is from about 0.5 mm to about 200 mm, from about 0.5 mm to about 100 mm, from about 0.5 mm to about 50 mm, from about 0.5 mm to about 25 mm, from about 0.5 mm to about 10 mm or from about 0.5 mm to about 5 mm. In other embodiments, the (solution volume probed)/(exposed surface area of the working electrode) for a working electrode is from about 5 mm to about 300 mm, from about 10 mm to about 300 mm, from about 25 mm to about 300 mm, from about 50 mm to about 300 mm, from about 100 mm to about 300 mm or from about 200 mm to about 300 mm.

When the working electrode is a micro electrode, typically a wire, the (solution volume probed)/(exposed surface area of the working electrode) for the working electrode may be from about 0.5 mm to about 1 mm, from about 1 mm to about 2 mm, from about 5 mm to about 10 mm or from about 25 mm to about 50 mm.

When the working electrode is a nano electrode, the (solution volume probed)/(exposed surface area of the working electrode) for the working electrode may be from about 100 mm to about 200 mm, from about 200 mm to about 300 mm or from about 100 mm to about 300 mm.

The working electrode of the wound dressing according to the present invention may comprise any material suitable for incorporation into a wound dressing according to the present invention and use for in a wound environment. Typically, the working electrode comprises at least one electrically conducting material but may in addition contain non-electrically conducting materials. In some embodiments, the working electrode comprises an electrically conducting ink or paste.

In embodiments, the working electrode is formed of any suitable metal, such as, Pt, Pd, Au, Ag or Cu. In some embodiments, the working electrode is formed of a metal alloy such as a copper alloy or stainless steel. In other embodiments, the working electrode is formed of a non-metallic material such as an organic conducting polymer or form of carbon. Such organic conducting polymers include polypyrrole, polyaniline or polythiophene sand their derivatives. Where the working electrode is formed of carbon, the carbon may be selected from carbon black, carbon nanotubes, graphite, graphene, carbon inks and carbon fibres. Preferably, the working electrode is formed of Pt.

It will be appreciated that the present invention is not limited to the use of metal based working electrodes. In some embodiments, the working electrode is a form of carbon.

The working electrode of the wound dressing according to the present invention may be any shape which is suitable for incorporation into a wound dressing and provides an exposed surface that is capable of detecting an electroactive species within the wound environment during use. In embodiments, the working electrode is any suitable geometric shape, for example, a cylinder, square prism, triangular prism, square pyramid or triangular prism. In some embodiments, the working electrode has a cross-sectional shape that is regular or irregular. The working electrode, in some instances, may also be elongate or planar.

Where the working electrode is elongate, the working electrode may be a wire. This includes free standing wires. In embodiments, the wire has a thickness of from about 0.1 μm to about 150 μm. The wire may have a thickness of from about 50 μm to about 150 μm or about 100 μm. The wire, in some embodiments, has a thickness of from about 20 μm to about 30 μm or about 25 μm. In other embodiments, the wire has a thickness of from about 1 μm to about 10 μm or about 5 μm. In further embodiments, the wire has a thickness of from about 0.1 μm to about 2 μm or about 1 μm. In embodiments where the working electrode is a wire, as described herein, the (solution volume probed)/(exposed surface area of electrode) may be from about 0.5 mm to about 100 mm.

Where the working electrode is elongate, the working electrode may be a micro-wire. In some embodiments, the micro-wire is printed onto a substrate layer. In such embodiments, the substrate (or substrate layer) may be any material as described herein. Where the micro-wire is printed onto a substrate layer, the substrate layer is formed of a polymer, more specifically a polyester such as polyethylene terephthalate (PET), Mylar (biaxially-oriented polyethylene terephthalate) or polybutylene terephthalate (PBT). In some embodiments, the substrate layer is formed of a cellulose based polymer, a polyimide (e.g. Kapton), a polystyrene, a polyamide or a polycarbonate. In some embodiments the substrate layer may be form of a paper or card pre-coated with a barrier layer

In embodiments, the micro-wire has a thickness of from about 0.1 μm to about 150 μm. The micro-wire may have a thickness of from about 50 μm to about 150 μm or about 100 μm. The micro-wire in some embodiments, has a thickness of from about 20 μm to about 30 μm or about 25 μm. In other embodiments, the micro-wire has a thickness of from about 1 μm to about 10 μm or about 5 μm. In further embodiments, the micro-wire has a thickness of from about 0.1 μm to about 2 μm or about 1 μm. In embodiments where the working electrode is a micro-wire, as described herein, the (solution volume probed)/(exposed surface area of electrode) may be from about 0.5 mm to about 100 mm.

In some embodiments, the micro-wire may be sandwiched or located between an insulating layer and substrate layer. In such embodiments, the insulating layer is formed of a dielectric material, for example a photo-, UV or thermally cured resist or dielectric ink. In embodiments, the insulating layer is formed of a dielectric material, for example epoxy based materials such as SU-8. In embodiments, the insulating layer is formed of PE773. The insulating layer may have a thickness of from about 1 μm to about 10 μm, from about 1 μm to about 8 μm, from about 1 μm to about 6 μm, or from about 1 μm to about 4 μm. Preferably, the insulating layer may have a thickness of about 2 μm, 3 μm, 4 μm or 5 μm.

Where the working electrode is elongate, the working electrode may be a micro-strip. In such embodiments, the substrate (or substrate layer) may be any material as described herein. Where the micro-strip is printed onto a substrate layer, the substrate layer is formed of a polymer, more specifically a polyester such as polyethylene terephthalate (PET), Mylar (biaxially-oriented polyethylene terephthalate) or polybutylene terephthalate (PBT). In some embodiments, the substrate layer is formed of a cellulose based polymer, a polyimide (e.g. Kapton), a polystyrene, a polyamide or a polycarbonate. In some embodiments the substrate layer may be form of a paper or card pre-coated with a barrier layer

In some embodiments, the micro-strip is printed onto a substrate. In embodiments, the micro-strip has a thickness of from about 0.1 μm to about 150 μm. The micro-strip may have a thickness of from about 50 μm to about 150 μm or about 100 μm. The micro-strip in some embodiments, has a thickness of from about 20 μm to about 30 μm or about 25 μm. In other embodiments, the micro-strip has a thickness of from about 1 μm to about 10 μm or about 5 μm. In further embodiments, the micro-strip has a thickness of from about 0.1 μm to about 2 μm or about 1 μm. In embodiments where the working electrode is a s micro-strip, as described herein, the (solution volume probed)/(exposed surface area of electrode) may be from about 0.5 mm to about 100 mm.

In some embodiments, the micro-strip may be sandwiched or located between an insulating layer and substrate layer. In such embodiments, the insulating layer is formed of a dielectric material, for example a photo-, UV or thermally cured resist or dielectric ink. In embodiments, the insulating layer is formed of a dielectric material, for example epoxy based materials such as SU-8. In embodiments, the insulating layer is formed of PE773. The insulating layer may have a thickness of from about 1 μm to about 10 μm, from about 1 μm to about 8 μm, from about 1 μm to about 6 μm, or from about 1 μm to about 4 μm. Preferably, the insulating layer may have a thickness of about 2 μm, 3 μm, 4 μm or 5 μm.

In some embodiments, the working electrode is a micro-wire or micro-strip electrode as described herein. A carbon micro-wire or carbon micro-strip working electrode may be formed by printing a carbon track/layer, typically having a thickness of from about 0.1 μm to about 150 μm, onto a suitable substrate (as described herein). This is used with a suitable counter and/or reference electrode. The carbon micro-wire/micro-strip working electrode may be covered with an insulating layer and then exposed in an analogous manner to that described for the electrode structure depicted in FIG. 3. That is, apertures may be cut down through the laminate structure of the carbon micro-wire or carbon micro-strip working electrode to expose a carbon micro-wire/micro-strip working electrode edge. This exposed carbon micro-wire/micro-strip may have a dimension of between 1 mm and 20 cm in length. A cross sectional view of a typical carbon micro-wire/micro-strip is shown in FIG. 22. It will be appreciated that the micro-wire or micro-strip electrode thus formed may be made from conductive materials other than carbon.

In embodiments, the working electrode may have a laminate structure. Typically, when the working electrode is planar, a laminate structure (or multi-layered structure) is utilised. It will be appreciated that where a laminate structure (or multi-layered structure) is utilised, the separate layers forming part of the structure may be secured together (e.g. chemically adhered or mechanically fixed) to form a single integrally formed structure. Alternatively, where a laminate structure (or multi-layered structure) is utilised, the layers forming part of the structure may not be secured together but freely arranged in a layered or laminate manner. The laminate structure of the working electrode of the invention may comprise a working electrode layer, insulating layer and substrate layer (as exemplified in FIG. 3). It will be appreciated that where the working electrode layer has a laminate structure, at least one surface of the working electrode layer must remain exposed to the wound environment during use to provide a sensing surface (e.g. contact surface) suitable for detecting an electroactive species within the wound environment.

It is contemplated that other layers may be present in the laminate structure of the working electrode. In some embodiments, the laminate structure may include one or more adhesive layers to bond or adhere the working electrode layer, insulating layer and substrate layer together to form the laminate structure of the electrode. In some embodiments, there may be multiple substrate layers and/or multiple adhesive layers.

In other embodiments, the working electrode layer is partly or fully sandwiched between the substrate layer and insulating layer to form the laminate structure. In such embodiments, the substrate layer may be bonded or adhered to one side of the working electrode layer whilst the insulating layer may be bonded or adhered to the other (opposing) side of the working electrode layer. In instances, the working electrode layer is substantially encapsulated by the insulating layer and the encapsulated working electrode layer is bonded or adhered to the substrate layer to form the laminate structure.

It will be appreciated that the laminate structure may includes at least one edge (or side surface) which leaves exposed part or all of the working electrode cross-section (e.g. insulating layer, working electrode layer and/or and substrate layer). This may result in a edge surface of the working electrode layer being left exposed to the wound environment during use in order to provide a sensing surface suitable for detecting an electroactive species within the wound environment. It will be appreciated that the electroactive species may undergo an electrochemical reaction (e.g. oxidation or reduction) at the sensing surface during use.

In embodiments, the laminate structure includes at least one aperture (or void) extending through the insulating layer to leave a surface of the working electrode layer exposed to the wound environment during use in order to provide a sensing surface suitable for detecting an electroactive species within the wound environment. It will be appreciated that the electroactive species may undergo an electrochemical reaction (e.g. oxidation or reduction) at the sensing surface during use.

In some embodiments, the working electrode is a planar laminate structure comprising a working electrode layer, insulating layer and substrate layer, wherein the working electrode layer is sandwiched between the substrate layer and insulating layer to form the laminate structure, the laminate structure includes at least one aperture extending through the insulating layer and the working electrode layer to leave a surface of the working electrode layer exposed to the wound environment during use in order to provide a sensing surface suitable for detecting the electroactive species within the wound environment, and the surface of the working electrode layer exposed comprises exposed edges which are formed at the periphery of the at least one aperture and the exposed edges have a thickness of from about 25 nm to about 75 nm.

In some embodiments, the working electrode is a planar laminate structure comprising a working electrode layer, insulating layer and substrate layer, wherein the working electrode layer is sandwiched between the substrate layer and insulating layer to form the laminate structure, the laminate structure includes at least one aperture extending through the insulating layer, the working electrode layer and part way through or all the way through the substrate layer to leave a surface of the working electrode layer exposed to the wound environment during use in order to provide a sensing surface suitable for detecting the electroactive species within the wound environment, and the surface of the working electrode layer exposed comprises exposed edges which are formed at the periphery of the at least one aperture and the exposed edges have a thickness of from about 25 nm to about 75 nm.

The at least one aperture may be of any suitable shape, for example, square, circular or triangular. Where the at least one aperture is square, the square aperture may have an edge dimension (e.g. length and width) of from about 1 μm to about 100 μm, from about 10 μm to about 100 μm, from about 40 μm to about 100 μm, from about 60 μm to about 100 μm, from about 80 μm to about 100 μm, from about 10 μm to about 80 μm, from about 10 μm to about 60 μm, from about 10 μm to about 40 μm or from about 10 μm to about 50 μm. Preferably, the square aperture may have an edge dimension of from about 20 μm to about 40 μm or about 30 μm. In some instances, the square aperture may have an edge dimension (e.g. length and width) of about 10 μm, about 30 μm or about 90 μm.

Where the at least one aperture is circular, the circular aperture may have a diameter of from about 1 μm to about 100 μm, from about 10 μm to about 100 μm, from about 40 μm to about 100 μm, from about 60 μm to about 100 μm, from about 80 μm to about 100 μm, from about 10 μm to about 80 μm, from about 10 μm to about 60 μm, from about 10 μm to about 40 μm or from about 10 μm to about 50 μm. Preferably, the circular aperture may have a diameter of from about 20 μm to about 40 μm or about 30 μm. In some instances, the circular aperture may have a diameter of about 10 μm, about 30 μm or about 90 μm.

In embodiments, where the working electrode layer is partly or fully sandwiched between the substrate layer and insulating layer to form the laminate structure, the at least one aperture is formed via an etching process whereby the at least one aperture is provided by etching away part of the insulating layer to leave a surface of the working electrode layer exposed to the wound environment during use in order to provide the sensing surface. In some embodiments, the at least one aperture is formed via an etching process whereby the at least one aperture is provided by etching away part of the insulating layer and part of the working electrode layer to leave edges of the working electrode layer, which are formed at the periphery of the at least one aperture, exposed to the wound environment during use. These exposed edges provide the sensing surface. An exemplary embodiment of a working electrode including exposed edges is depicted in FIG. 3 and described herein. Suitable etching processes are well known in the art.

The exposed edges as described herein comprise a thickness (or height) dimension of less than 1000 nm. In embodiments, the exposed edges comprise a thickness of from about 5 nm to 200 nm, from about 25 nm to about 200 nm, from about 50 nm to about 200 nm, from about 100 nm to about 200 nm, from about 150 nm to about 200 nm, from about 5 nm to about 150 nm, from about 5 nm to about 100 nm, from about 5 nm to about 50 nm or from about 5 nm to about 25 nm. Preferably, the exposed edges comprise a thickness of from about 25 nm to about 75 nm or from about 35 nm to about 65 nm or from about 45 nm to about 55 nm. In embodiments, the exposed edges comprise a thickness of about 50 nm.

It will be appreciated that the at least one aperture may comprise an array of apertures. The array of apertures may be formed in a uniform pattern on the working electrode. For example, the array of apertures may be formed in a grid formation on the working electrode, such as exemplified in FIG. 4.

In some embodiments, the array of apertures include an interval (or spacing distance) between adjacent apertures of from about 10 μm to about 200 μm, from about 10 μm to about 100 μm, from about 40 μm to about 100 μm, from about 60 μm to about 100 μm, from about 80 μm to about 100 μm, from about 10 μm to about 80 μm, from about 10 μm to about 60 μm, from about 10 μm to about 40 μm or from about 20 μm to about 60 μm. Preferably, the array of apertures may include an interval between adjacent apertures of about from about 30 μm to about 50 μm or about 40 μm. In some instances, the array of apertures may include an interval between adjacent apertures of about 10 μm, about 30 μm or about 90 μm. Without being bound by theory it will be appreciated that by ensuring that the apertures include intervals of a suitable size, the array of apertures do not approximate to a macro electrode and avoid the issues discussed herein in relation to the small double layer charging and signal to noise ratio issues associated with such macro electrode based wound dressings.

The array of apertures as described herein may comprise from about 1000 apertures to about 100000 apertures, from about 1000 apertures to about 10000 apertures, from about 2000 apertures to about 8000 apertures or from about 4000 apertures to about 6000 apertures. Preferably, the array of apertures as described herein may comprise from about apertures 5000 to about apertures 7000 or from about 5500 apertures to about 6500 apertures.

In embodiments, the working electrode layer is formed of any suitable metal, such as, Pt, Pd, Au, Ag or Cu. In some embodiments, the working electrode layer is formed of a metal alloy such as a copper alloy or stainless steel. In some embodiments, the working electrode layer is formed of a non-metallic material such as an organic conducting polymer. Such organic conducting polymers include polypyrrole, polyaniline or carbon. Where the working electrode comprises carbon, the carbon may be selected from any conductive form of carbon such as carbon black, carbon nanotubes, graphite, graphene, carbon inks and carbon fibres. Preferably, the working electrode layer is formed of Pt.

The working electrode layer may have a thickness of from about 5 nm to 200 nm, from about 25 nm to about 200 nm, from about 50 nm to about 200 nm, from about 100 nm to about 200 nm, from about 150 nm to about 200 nm, from about 5 nm to about 150 nm, from about 5 nm to about 100 nm, from about 5 nm to about 50 nm or from about 5 nm to about 25 nm. Preferably, the working electrode layer has a thickness of from about 25 nm to about 75 nm or from about 35 nm to about 65 nm or from about 45 nm to about 55 nm. In embodiments, the working electrode layer has a thickness of about 50 nm.

In some embodiments, the substrate layer is formed of a polymer, more specifically a polyester such as polyethylene terephthalate (PET), Mylar (biaxially-oriented polyethylene terephthalate) or polybutylene terephthalate (PBT). In some embodiments, the substrate layer is formed of a cellulose based polymer, a polyimide (e.g. Kapton), a polystyrene, a polyamide or a polycarbonate. In some embodiments the substrate layer may be form of a paper or card pre-coated with a barrier layer.

In embodiments, the insulating layer is formed of a dielectric material, for example a photo-, UV or thermally cured resist or dielectric ink. In embodiments, the insulating layer is formed of a dielectric material, for example epoxy based materials such as SU-8. In embodiments, the insulating layer is formed of PE773. The insulating layer may have a thickness of from about 1 μm to about 10 μm, from about 1 μm to about 8 μm, from about 1 μm to about 6 μm, or from about 1 μm to about 4 μm. Preferably, the insulating layer may have a thickness of about 2 μm, 3 μm, 4 μm or 5 μm.

It will be appreciated that when the working electrode is planar, in particular when the working electrode has a laminate structure as described herein, that the (solution volume probed)/(exposed surface area of electrode) is from about 100 mm to about 200 mm.

The wound dressing according to the present invention may comprise a laminate structure comprising a backing layer, support layer and wound contact layer (as exemplified in FIGS. 1 and 5-9). It is contemplated that other layers may be present in the laminate structure of the wound dressing as described herein. In some embodiments, the laminate structure may include one or more adhesive layers to bond or adhere the backing layer, support layer, wound contact layer and/or any other layer together to form the laminate structure. In other embodiments, the laminate structure may include one or more dressing layers (or absorbent layers) suitable for receiving and/or retaining fluid (e.g. wound exudate) present within the wound environment during use. In some embodiments, the dressing layer is carboxymethyl cellulose; optionally loaded with silver. In other embodiments, the dressling layer may be a hydrocolloids, hydrogels, alginates, acrylate/polyurethane films/foams, collagen, silicone, or any combination thereof. The dressing layer may optionally be loaded with at least one anti-microbial or anti-biofilm agent.

In some embodiments, the electrochemical sensor is sandwiched between a dressing layer, such as carboxymethyl cellulose, and a non-woven fabric layer (for example non-woven polypropylene fabric).

The support layer of the wound dressing of the present invention may be any material suitable for holding or housing the electrochemical sensor within the overall structure of the wound dressing. In some embodiments, the backing layer may be any material suitable for supporting a working electrode and/or a counter/reference electrode within the overall structure of the wound dressing. It will be appreciated that the electrochemical sensor, working electrode and/or counter/reference electrode may be attached or secured to the support layer by any suitable means. In some embodiments, the electrochemical sensor, working electrode and/or counter/reference electrode is adhered to the support layer. In other embodiments, the electrochemical sensor, working electrode and/or counter/reference electrode may form part of the support layer's structure. For example, where the working electrode is a wire and the support layer is a wire mesh, the working electrode may integrally form part of the wire mesh structure. In some embodiments, the working electrode is a wire and forms part of an open mesh together with the support layer. In other embodiments, the working electrode is a wire and forms part of a series of parallel wires held within the support layer, wherein the support layer is optionally a non-conducting support mesh. One advantage associated with the use of the wire electrodes as described herein is the electroactive species present in the wound environment may diffuse to the electrode surface from a radial area surrounding the working electrode wire.

The backing layer of the wound dressing of the present invention may be any material suitable for protecting the wound dressing of the present invention from the external environment during use (i.e. when attached to the wound). Suitable backing layers include a polyurethane backing film or polyethylene backing film. In some embodiments, the backing layer allows fluid (e.g. water vapour) to flow through the backing layer from the wound (to benefit breathability) but does not allow liquid water to move through the backing layer in the direction of the wound (to benefit waterproofing).

The wound contact layer of the wound dressing of the present invention may be any material which is suitable for being in direct contact with or proximal to the wound during use. It will be appreciated that the wound contact layer may be permeable to any electroactive species as described herein to allow for the passing (or flow) of the electroactive species from the wound to the electrochemical sensor within the wound environment during use. The wound contact layer, in some embodiments, will be porous to the fluid which contains the electroactive species within the wound environment. In particular, the wound contact layer may allow the flow of fluid which contains the electroactive species (e.g. wound exudate) from within the wound through the wound contact layer and to the electrochemical sensor within the wound dressing for analyte detection. The wound contact layer, in some embodiments, may include an active agent as described herein. For example, the wound contact layer may include an anti-microbial or anti-biofilm agent. This active agent may be identical to the electroactive species being detected or different to the electroactive species.

In some embodiments, the wound dressing of the present invention comprises a reference electrode. Suitable reference electrodes will be known to the skilled person and may vary depending on the nature of the electrochemical detection technique being performed using the electrochemical sensor as described herein. The reference electrode may be any material which remains stable and its electrochemical potential remains constant when operating the electrochemical sensor in order to enable detection of the electroactive species at the working electrode within the wound environment during use. In embodiments, the reference electrode may be silver or silver/silver chloride. In addition, the reference electrode may be exposed or coated with a protective semi-permeable polymer layer or composite layer designed to maintain a desired ionic environment for the reference electrode.

It will be appreciated that electrochemical sensors may be operated as 2-electrode sensors comprising a working electrode and a counter/reference electrode or as 3-electrode sensors comprising working electrode, and separate reference and counter electrodes. In the embodiments described herein, the electrochemical sensors are operated as 2-electrode sensors comprising a working electrode and a counter/reference electrode. This has the benefit of preventing potential damage to the sensor which can occur in a 3-electrode sensor if the reference electrode is the last electrode to be exposed to the wound fluid during hydration. It will be known to a skilled person however that the sensors described herein could be implemented in a 3-electrode configuration with the placement of a separate reference electrode in very close proximity to the working electrode and location of the counter electrode as is described for the counter/reference electrode herein.

The working electrode may be attached or secured to a wound facing surface of the support layer and/or the counter/reference electrode is attached or secured to a wound facing surface of the support layer. In some embodiments, the working electrode is attached or secured to a wound facing surface of the support layer and the counter/reference electrode is attached or secured to a non-wound facing surface (opposite to the wound facing surface) of the support layer. In other embodiments, the counter/reference electrode is attached or secured to a wound facing surface of the support layer and the working electrode is attached or secured to a non-wound facing surface (opposite to the wound facing surface) of the support layer.

In some embodiments, the working electrode and the counter/reference electrode are physically separated (electrically isolated) from one another within the wound dressing structure to prevent direct electrical contact, whilst maintaining ionic conduction through the wound fluid. This may be achieved by positioning the working electrode and the counter/reference electrode on separate layers of the dressing structure to provide the electrical isolation. Additionally, or alternatively, the working electrode and/or the counter/reference electrode may be coated with an electrically insulating material. It will be appreciated that the insulating material will be porous to the electroactive species which is to be detected during use and allow ionic conduction. In further embodiments, such as the embodiment exemplified in FIG. 7, the wound dressing comprises a spacing layer which provides an electrically insulating barrier between the working electrode and the counter/reference electrode thus preventing direct electrical contact between the two electrodes.

In embodiments, the wound dressing according to the present invention is also configured to release and then monitor the concentration of any electroactive species as described herein (e.g. anti-microbial or anti-biofilm agent) in or on the wound or in its surrounding environment while the wound dressing is in use and applied to the body of a human or animal subject. By this, it is meant that the wound dressing is capable of administering an electroactive species (e.g. an active agent) into the wound environment and subsequently detect and monitor the concentration of that electroactive species during use. The wound dressing may further comprise an active release electrode suitable for controlling the release of electroactive species into the wound environment and an active release layer which contains and releases the electroactive species. The active release electrode and the active release layer may be electrically isolated from other components within the wound dressing, in particular, the active release electrode and the active release layer may be electrically isolated from the working electrode and the counter/reference electrode. This may be achieved using one or more spacing layers.

The active release layer may be formed on the surface of the active release electrode and the electroactive species may form part of the active release layer. The active release electrode may be configured to cause the release of the electroactive species from the active release layer into the wound environment via an electrochemical reaction (e.g. oxidation or reduction) which takes place between the active release electrode and active release layer during use. In such embodiments, the working electrode is configured to detect (or sense) the concentration of the electroactive species being released within the wound environment.

The wound dressing of the present invention may additionally comprise a wound status sensor configured to monitor a suitable wound status parameter (e.g. temperature, impedance, pH, oxygen levels, moisture content, toxin levels (e.g. bacterial endo and exotoxins levels), signalling compounds, enzyme (e.g. amylase) and/or growth factors being released by the wound, outer membrane proteins and quorum sensing molecules).

Where the wound dressing includes the wound status sensor, the active release electrode, active release layer and the wound status sensor may be in communication such that an electroactive species can be released from the active release layer via an electrochemical reaction on the active release electrode in response to a change in the wound status parameter monitored by wound status electrode. The level of the electroactive species present in the wound environment is then detected by the working electrode.

In embodiments, the wound status sensor may comprise an ion selective electrode, amperometric electrochemical sensor, coulometric electrochemical sensor, anodic stripping voltametric sensor, capacitive sensor, electrochemical impedance sensor, optical sensor, piezoelectric sensor and colourimetric sensor.

The working electrode, in some embodiments, includes a coating on its surface which is permeable to an electroactive species to be detected. In some embodiments, the permeability of the coating is selective for a predetermined target electroactive species to be detected. For instance, the coating may be chosen so as to be permeable to the target species and relatively impermeable (e.g. due to size exclusion or polarity, etc.) to another electroactive species that is not desired to be detected. The permeability of the coating to the substance may be tuned depending on the assumed levels of electroactive species to be detected in the wound environment. For instance, a coating with a higher permeability to a given electroactive species may be used (when assumed concentration of the species is low) or with a lower permeability to the given species depending on the assumed concentration of the electroactive species within the wound environment. The electroactive species may be any material (e.g. an enzyme) which is released by the wound within the wound environment during use.

The working electrode, in some embodiments, includes a coating on its surface which is permeable to an electroactive species to be detected. In such embodiments, the permeability of the coating is increased or decreased depending on the concentration level of substance within the wound environment. The substance may be any material (e.g. an enzyme) which is released by the wound within the wound environment during use. The substance may increase the permeability of the coating by breaking down the coating or decrease the permeability of the coating by deposition on the coating. It will be appreciated that the substance may be different to the electroactive species. In some embodiments, the substance is an enzyme (e.g. a protease) that increases the permeability of a polypeptide coating formed on the surface of the working electrode by digesting the polypeptide.

In embodiments of the second and fourth aspect of the present invention, operating the electrochemical sensor comprises detecting the electroactive species by anodic stripping voltammetry, amperometry, potentiometry, electrochemical impedance analysis (e.g. electrochemical impedance spectroscopy) or derivative techniques thereof, preferably, wherein the electrochemical sensor detects the electroactive species by anodic stripping voltammetry. In other embodiments, the operating of the electrochemical sensor comprises detecting an electroactive species as a result of deposition or breakdown of a layer or coating on the working electrode surface. For example, the electrochemical sensor may include a working electrode having a polypeptide coating. In use, the polypeptide coating is broken down and its permeability to the electroactive species altered by an enzyme (e.g. protease) released from the wound. In further embodiments, the operating the electrochemical sensor comprises detecting an electroactive species that is either generated by, or consumed by, a biofilm or bioburden within the wound environment. For example, the biofilm may produce ammonia which can be detected or the biofilm may consume an oxygen species (e.g. molecular oxygen) in the wound environment which may also be detected.

The method according to the second and fourth aspect of the present invention may further comprises administering an anti-microbial agent or anti-biofilm agent into the wound in response to the level of electroactive species detected in the wound environment; optionally wherein the electroactive species detected is the anti-microbial agent or anti-biofilm agent. The anti-microbial agent and/or anti-biofilm agent may be a metal selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr. In embodiments, the anti-microbial agent and/or anti-biofilm agent is Ag. In some embodiments, the anti-microbial agent or anti-biofilm agent may, for example, be a metal ion selected from the group consisting of Ag, Al, Au, Ba, Bi, TI, Ce, Co, Cu, Fe, Ga, Ge, Ir, Mo, Rh, Ru, Sb, Se, Sn, Sr, Ti, W and Zn ions. In some embodiments of the second and fourth aspect of the present invention, the electroactive species is generated by a biofilm or bioburden present within the wound environment.

The method may include using the electrochemical sensor to detect an electroactive species within the wound environment at concentration levels of less than about 10 parts per million (ppm) of the electroactive species. In some embodiments, the electrochemical sensor detects an electroactive species within the wound environment at concentration levels of less than about 5 ppm of the electroactive species. In some embodiments, the electrochemical sensor detects an electroactive species within the wound environment at concentration levels of at least about 10 parts per billion (ppb) of the electroactive species.

In embodiments, the method may include generating an output signal representative of the level of the electroactive species detected.

The electrochemical sensor may be as defined according to any embodiment herein.

The method may for instance include use of a working electrode comprising a coating, wherein the permeability of the coating is selected for a predetermined target electroactive species to be detected. For instance, the coating may be chosen so as to be permeable to the target species and relatively impermeable (e.g. due to size exclusion or polarity, etc.) to another electroactive species that is not desired to be detected. In embodiments, the permeability of the coating to the substance is tuned depending on the assumed levels of the electroactive species to be detected in the wound environment.

For instance, a coating with a higher permeability to a given electroactive species may be used (when assumed concentration of the species is low) or with a lower permeability to the given species depending on the assumed concentration of the electroactive species within the wound environment.

In embodiments of the third aspect of the present invention, the use comprises operating the electrochemical sensor to detect the electroactive species. For example, this can be done by anodic stripping voltammetry, amperometry, potentiometry, electrochemical impedance analysis (e.g. electrochemical impedance spectroscopy) or derivative techniques thereof, preferably, wherein the electrochemical sensor detects the electroactive species by anodic stripping voltammetry. In other embodiments, the operating the electrochemical sensor comprises detecting an electroactive species as a result of deposition or breakdown of a layer or coating on the working electrode surface.

In further embodiments, the operating of the electrochemical sensor comprises detecting an electroactive species that is either generated by, or consumed by, a biofilm or bioburden within the wound environment.

The use according to the third aspect of the present invention may further comprise administering an anti-microbial agent or anti-biofilm agent into the wound in response to the level of electroactive species detected in the wound environment; optionally wherein the electroactive species detected is the anti-microbial agent or anti-biofilm agent. The anti-microbial agent and/or anti-biofilm agent may be a metal selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr. In embodiments, the anti-microbial agent and/or anti-biofilm agent is Ag. In some embodiments, the anti-microbial agent or anti-biofilm agent may, for example, be a metal ion selected from the group consisting of Ag, Al, Au, Ba, Bi, TI, Ce, Co, Cu, Fe, Ga, Ge, Ir, Mo, Rh, Ru, Sb, Se, Sn, Sr, Ti, Wand Zn ions. In some embodiments of the third aspect of the present invention, the electroactive species is generated by a biofilm or bioburden present within the wound environment.

The use may include using the electrochemical sensor to detect an electroactive species within the wound environment at concentration levels of less than about 10 parts per million (ppm) of the electroactive species. In some embodiments, the electrochemical sensor detects an electroactive species within the wound environment at concentration levels of less than about 5 ppm of the electroactive species.

In embodiments, the use may include generating an output signal representative of the level of the electroactive species detected.

The use may for instance include use of a working electrode comprising a coating, wherein the permeability of the coating is selected for a predetermined target electroactive species to be detected. For instance, the coating may be chosen so as to be permeable to the target species and relatively impermeable (e.g. due to size exclusion or polarity, etc.) to another electroactive species that is not desired to be detected. In embodiments, the permeability of the coating to the substance is tuned depending on the assumed levels of the electroactive species to be detected in the wound environment.

For instance, a coating with a higher permeability to a given electroactive species may be used (when assumed concentration of the species is low) or with a lower permeability to the given species depending on the assumed concentration of the electroactive species within the wound environment.

In a fifth aspect of the present invention there is a wound dressing comprising an active agent and provided with an electrochemical sensor configured to sense a level of the active agent while the wound dressing is in situ, and to output a signal representative of the sensed level of the active agent.

In embodiments of the fifth aspect of the present invention, the electrochemical sensor comprises electrodes on a substrate and in which the electrodes are on a side of the substrate which faces away from or facing the wound when the dressing is in situ.

According to the fifth aspect of the present invention or embodiments thereof, the active agent comprises one or more of an anti-microbial (including silver, copper, cerium, zinc, rubidium, iodine, PHMB, octenidine, chlorhexidine, benzalkonium chloride, quaternary ammonium compounds, peracetic acid, gold surfactant (including the above cationic, anionic and neutral surfactants eg poloxamers)), chelating agents (including EDTA (di sodium, tri sodium, tetra sodium), EGTA, citrates, polyphosphates, citrates or combinations thereof etc), an antibiofilm agent, a growth factor, an anti-inflammatory, a peptide, an antifungal, antiviral, an enzyme, an anti-biotic.

In embodiments of the fifth aspect of the present invention, the electrochemical sensor is configured to detect concentration of the active agent in a solution.

In some embodiments of the fifth aspect of the present invention, the electrochemical sensor is configured to detect concentration of the active agent in a solution comprising bodily exudate and/or comprising liquid carried by the wound dressing.

In embodiments of the fifth aspect of the present invention, the electrochemical sensor comprises one or more of a mass balance, an optical sensor, a resistive sensor and a magnetic sensor.

In embodiments of the fifth aspect of the present invention, the electrochemical sensor comprises an electrode which is a micro electrode as described herein or a nano electrode as described herein configured to be exposed to a solution containing the active agent. In such embodiments, the electrode is connected to measurement electronics configured to operate the sensor by anodic stripping.

In embodiments of the fifth aspect of the present invention, the dressing comprises a laminate structure comprising a conductive layer (or working electrode layer) faced on either side by an insulating layer (e.g. a dielectric material) and etched to expose an edge of the working electrode layer, the said edge forming an electrode sensing surface. In embodiments of the fifth aspect of the present invention, the electrochemical sensor comprises a voltammetric sensor.

In embodiments of the fifth aspect of the present invention, the electrode is connected to measurement electronics configured to operate the sensor by anodic stripping.

According to the any aspect of the present invention or embodiments thereof, the electrochemical sensor is configured to detect Ag ion concentration and/or Cu ion concentration.

In embodiments, the electrochemical sensor has a sensor which is attached to or incorporated into the wound dressing or into an associated adhesive layer or support layer.

In embodiments, the electrochemical sensor is configured to be wetted by bodily exudate or another solution in use.

In embodiments, the electrochemical sensor is configured to be wetted by liquid carried by the wound dressing.

In embodiments, the electrochemical sensor is mounted on an internal or external surface of the wound dressing directed away from the subject on which the dressing is applied when the dressing is in situ.

According to the any aspect of the present invention or embodiments thereof, the electrochemical sensor comprises a first unit comprising a sensor electrically connected to a second unit housing measurement electronics. The electrical connection may incorporate a releasable connector.

In embodiments, the electrochemical sensor is provided with a wireless transceiver for transmitting output data representative of the sensed level of the active agent to a separate processing device.

In embodiments, the electrochemical sensor is configured to be powered through the transceiver by energy harvested from an interrogating electromagnetic field.

In embodiments, the electrochemical sensor is configured to be powered by a battery in which bodily exudate serves as electrolyte.

In a sixth aspect of the present invention there is provided a wound dressing monitoring system comprising a wound dressing according to the first aspect and any embodiments thereof or fifth aspect and any embodiments thereof and a processing device configured to receive output data from the wound dressing.

According to embodiments of the sixth aspect of the present invention, the processing device is configured to determine from the output data whether the level of the active agent is below a threshold. The processing device may be configured to provide a warning to a user in the event that the level of the active agent is determined to be below the threshold.

In a seventh aspect of the present invention there is provided a electrochemical sensor comprising a sensor configured to be mounted in or on a wound dressing, to sense a level of an active agent while the wound dressing is in situ, and to output a signal representative of the sensed level of the active agent.

In embodiments of the seventh aspect of the present invention, the sensor comprises electrodes on a substrate and in which the electrodes are on a side of the substrate which faces away from the subject on which the dressing is applied when the dressing is in situ. The active agent may comprise one or more of an anti-microbial, an anti-biofilm agent, a growth factor, an anti-inflammatory, a peptide, an anti-fungal and an anti-biotic.

In some embodiments of the first aspect of the present invention, the working electrode has a planar laminate structure comprising a working electrode layer, insulating layer and substrate layer, wherein the working electrode layer is sandwiched between the substrate layer and insulating layer to form the laminate structure, and the substrate layer is bonded or adhered to one side of the working electrode layer whilst the insulating layer is bonded or adhered to the other (opposing) side of the working electrode layer, wherein the laminate structure includes at least one aperture (or void) extending through the insulating layer to leave a surface of the working electrode layer exposed to the wound environment during use in order to provide a sensing surface suitable for detecting an electroactive species within the wound environment, wherein the at least one aperture is formed via an etching process whereby the at least one aperture is provided by etching away part of the insulating layer and part of the working electrode layer to leave edges of the working electrode layer, which are formed at the periphery of the at least one aperture, exposed to the wound environment during use, wherein the exposed edges comprise a thickness of from about 25 nm to about 75 nm (e.g. about 50 nm). In these embodiments, the at least one aperture comprises an array of apertures wherein the array of apertures include an interval between adjacent apertures of about from about 30 μm to about 50 μm (e.g. about 40 μm), wherein the surface area of the working electrode that is exposed to the wound environment during use is from about 0.001 mm² to about 0.04 mm², and wherein the (solution volume probed)/(exposed surface area of the working electrode) for the working electrode is from about 100 mm to about 300 mm (e.g. from about 125 mm to about 175 mm).

### DESCRIPTION OF FIGURES

The invention will be further described, by way of example only, with reference to the accompanying Figures.

FIG. 1 is a schematic representation of an electrochemical sensor arrangement according to the present invention.

FIG. 2 is a block diagram illustrating the major functional units of a sensor and reporting arrangement according to the present invention.

FIG. 3 is a cross sectional schematic of an electrode arrangement according to the present invention.

FIG. 4 is a plan view of an electrode arrangement according to the present invention.

FIG. 5 is a schematic representation of a wound dressing arrangement including an electrochemical sensor according to the present invention.

FIG. 6 is a schematic representation of a wound dressing arrangement including an electrochemical sensor according to the present invention.

FIG. 7 is a schematic representation of an alternative wound dressing arrangement including an electrochemical sensor according to the present invention wherein the counter/reference electrode is electrically isolated from the working electrode.

FIG. 8 is a schematic representation of a wound dressing arrangement including an electrochemical sensor according to the present invention further including an active release electrode.

FIG. 9 is a schematic representation of a wound dressing arrangement including an electrochemical sensor according to the present invention further including an active release electrode and wound sensing electrode.

FIG. 10 shows a graph plotting the results of cyclic voltammetry experiments conducted using a platinum macro electrode, a 100 μm platinum wire electrode and a 25 μm platinum wire electrode as described in the “experimental data” section provided herein.

FIG. 11 shows a graph plotting the results of anodic stripping voltammetry experiments conducted in seven Mueller-Hinton Broth II (MHB2) solutions containing differing concentrations of added Ag⁺ ions using a Pt Nanoband Electrode as described in the “experimental data” section provided herein.

FIG. 12 shows an expanded version of FIG. 11 focusing on the section of the graph which illustrates the stripping step of the anodic stripping voltammetry experiments conducted.

FIG. 13 shows the oxidation charge during the anodic stripping voltammetry experiments of FIGS. 11 & 12 plotted as a function of the Ag⁺ ion concentration.

FIG. 14 shows the results of bacterial proliferation assays in Phosphate buffered saline (PBS), Silver Nitrate (NaNO3), Simulated wound Fluid (SWF) and Mueller Hinton Broth II (MHB2) solutions as a function of the amount of added Ag⁺ ions as described in the “experimental data” section provided herein.

FIG. 15 shows the results of optical density measurements in Phosphate buffered saline (PBS), Silver Nitrate (NaNO3), Simulated wound Fluid (SWF) and Mueller Hinton Broth II (MHB2) solutions as a function of the amount of added Ag⁺ ions as described in the “experimental data” section provided herein.

FIG. 16 shows the oxidation charge during anodic stripping voltammetry experiments conducted in Phosphate buffered saline (PBS), Silver Nitrate (NaNO3) and Simulated wound Fluid (SWF) solutions as a function of the Ag⁺ ion concentration using a Pt Nanoband Electrode as described in the “experimental data” section provided herein.

FIG. 17 shows the same results of FIG. 16 on a logarithmic scale.

FIG. 18 shows the oxidation charge during anodic stripping voltammetry experiments conducted in simulated wound fluid (SWF) plotted as a function of the Ag⁺ ion concentration using a 25 μm platinum wire electrode as described in the “experimental data” section provided herein.

FIG. 19 shows the results of anodic stripping voltammetry experiments involving multiple anodic steps using a nanoband electrode. The experiments were conducted in both simulated wound fluid (SWF) and simulated wound fluid with added silver ions as described in the “experimental data” section provided herein.

FIG. 20 shows an experiment using a silver release electrode and a Pt nanoband sensor. The experiment is designed to both generate and detect Ag⁺ ions as described in the “experimental data” section provided herein.

FIG. 21 shows measurement of iodine in phosphate buffer saline using a Macro electrode versus a platinum based nanoband electrode.

FIG. 22 is a schematic representation of a micro-wire/micro-strip electrochemical sensor arrangement according to the present invention.

FIG. 23 shows a graph plotting the results of cyclic voltammetry experiments conducted using a carbon micro-wire electrode with ferrocene carboxylic acid versus a carbon macro electrode as described herein.

FIG. 24 shows measurement of iodine in phosphate buffer saline using a macro printed carbon electrode (left hand side) as described herein versus a carbon micro-wire based electrode (right hand side).

FIG. 25 shows the calibration curve for a carbon micro-wire sensor with iodine.

FIG. 26 shows a schematic of a nanoband electrode sensor incorporated within a wound dressing arrangement.

FIG. 27 shows a photograph of the wound dressing sensor illustrated in FIG. 26 mounted within a flow cell.

FIG. 28 shows a plot of an anodic stripping charge for a nanoband electrode sensor incorporated within a wound dressing arrangement subjected to simulated wound fluid flow.

### DETAILED DESCRIPTION OF THE INVENTION

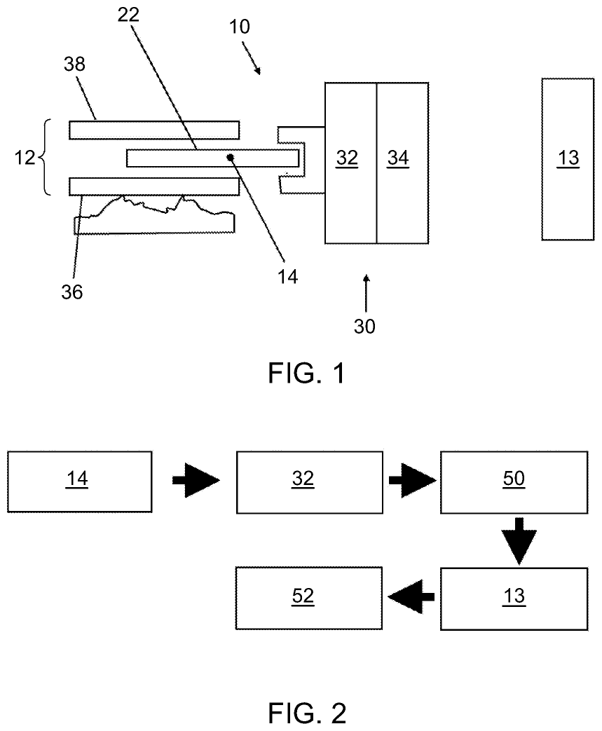

Referring to FIG. 1, there is depicted an electrochemical sensor 10 (comprising components 14, 32 and 34) according to a preferred embodiment of the present invention. The electrochemical sensor 10 forms part of a wound dressing 12 and is configured to monitor concentration of an anti-microbial or anti-biofilm agent in or on the wound dressing 12 or in its surrounding environment (i.e. within the wound environment) while the wound dressing is in use and applied to the body of a patient. The electrochemical sensor 10 is self-contained and is configured to transmit measurement data wirelessly to a separate processing device 13.

The electrochemical sensor 10 of this embodiment is operable to those skilled in the art to perform anodic stripping voltammetry. It will be appreciated that anodic stripping voltammetry is not the only technique that could be employed by electrochemical sensor 10. In this embodiment, the electrochemical sensor 10 is usable to monitor the concentration of electroactive metal ions, and more specifically of silver ions.

The electrochemical sensor 10 uses a micro- or nano-electrode arrangement as described herein. These have advantages over larger electrodes in that their small size leads to high current density even at low power. Depletion of the electroactive species in the region of the electrode can also be low, and mass transport effects which typically limit the performance of macro electrodes, are avoided.

As illustrated in FIGS. 1, 3 and 4 the electrochemical sensor contains an electrode component 14, which in preferred embodiments has a planar laminate structure incorporating a thin electrically conductive layer 16 (i.e. working electrode layer) whose upper surface and lower surface are covered with an insulating layer 22, and a substrate layer 20, respectively. The working electrode layer 16 includes exposed edges 18 which form the exposed sensing surfaces of the electrode itself. In the present embodiment the working electrode layer 16 is platinum and is disposed upon a substrate 20, which in the present embodiment is flexible and formed from a polymer, more specifically polyester.

Other substrate materials, flexible or rigid, may be adopted in other embodiments. The face of the working electrode layer 16 directed away from the substrate layer 20 carries the insulating layer 22. The insulating layer may be formed of a dielectric material. The insulating layer is advantageously able to perform two functions; it electrically insulates the face of the working electrode layer 16 and also has the ability to serve during manufacture as a resist, defining suitable areas to be etched. The material used for the insulating layer (resist) used in the present embodiment may be epoxy based, e.g. it may be what is known in the art as SU-8. Other suitable materials may be substituted, such as, PE773. The functions of etch resist and electrical insulation may in other embodiments be performed by two separate layers. In the example illustrated in FIGS. 1, 3 and 4, the dielectric insulating layer 22 is absent during manufacture from selected regions which form a grid of square shaped apertures 24. Other grid patterns and aperture shapes may be adopted. After deposition of the layers making up the structure of the electrode component 14, which may for example be by casting, spinning, sputtering, growth or deposition, the apertures 24 are then formed by etching to form voids in the selected regions. Suitable etching processes are well known in the art. The etching process removes the insulating layer 22 and the working electrode layer 16 to form apertures 24 have exposed edges 18 at the periphery of each aperture 24. The exposed edges provide the sensing surface for the working electrode layer 16.

The thickness of the working electrode layer 16, and hence the thickness (labelled as “D”) or height of the exposed edges 18, is in the order of 50 nm. The insulating layer 22 may have a thickness of around 3 μm. The square apertures 24 have side or edge length dimensions of 30 μm and are in embodiments arranged at intervals of 40 μm over a 2 mm square area.

In order to perform anodic stripping voltammetry, the electrochemical sensor 10 may additionally comprise a counter/reference electrode (not shown). The counter electrode and the reference electrode are formed by the same component but they may in other embodiments be separately formed. The counter/reference electrode comprises silver and silver chloride, in this example. Other suitable materials may be used.

The illustrated electrode component 14 is suitable for performance of anodic stripping voltammetry measurements without need of hydrodynamic control. The electrochemical sensor 10 may be configured to measure anti-microbial or anti-biofilm levels down to 0.01 parts per billion.

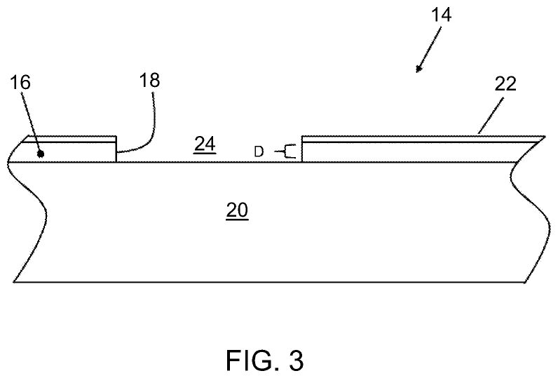

As illustrated in FIG. 1, the electrochemical sensor 10 includes two units 14, 30 which are electrically connected, in this embodiment by electrical wiring. Structure 14 is an electrode component containing a working electrode 16, 18 according to the invention (see FIG. 3) and is coupled to or incorporated within the wound dressing 12. Electrode component 14 itself is small enough to be accommodated or incorporated in a wound dressing 12. The second unit 30 houses associated driver electronics 32 and a power source in the form of a battery 34 and requires no physical connection to a further system, so that the patient is not discomforted by trailing wires leading to an external fixed unit.

The unit 30 is configured to (i) drive the working electrode 16, 18 of the electrode component 14 to obtain measurements of electroactive species within the wound dressing environment, (ii) receive an output signal from the working electrode and (iii) transmit the resultant measurement data to a separate processing device 13.

In the depicted embodiment, the driver electronics 32 used may be partly analogue and partly digital, comprising at least one microprocessor. The analogue signal from the working electrode may be applied to a suitable analogue front end which provides and controls the drive signals applied to the electrodes (a suitable commercial example is the LMP91000 from Texas Instruments) and which interfaces with a microcontroller. The driver electronics—and the second unit 30 as a whole—can be small enough and light enough to be suitable for affixing to the exterior of the wound dressing itself, to a wound dressing retainer system, or to the patient 10 or their clothes, without undue stress or inconvenience to the patient.

Wring from the electrode component 14 to the second unit 30 incorporates a releasable connector comprising a first connector part (plug) wired to the working electrode 16,18 and a second connector part (socket) wired to the electronics of the second unit, so that (a) if the electrode component 14 is disposed of after use, the second unit 30 can be connected to a fresh electrode component and re-used and (b) the wound dressing 12 can be applied without the encumbrance of the second unit 30, which is then connected and secured in place after application of the dressing. A simple push-fit connector is used. The wiring from the electrode component 14 needs in practice to be led out of any wound dressings so that the first connector part is accessible.

In the present embodiment the electrode component 14 is disposed inside the wound dressing 12 with the exposed working electrode edges 18 facing away from the wound.

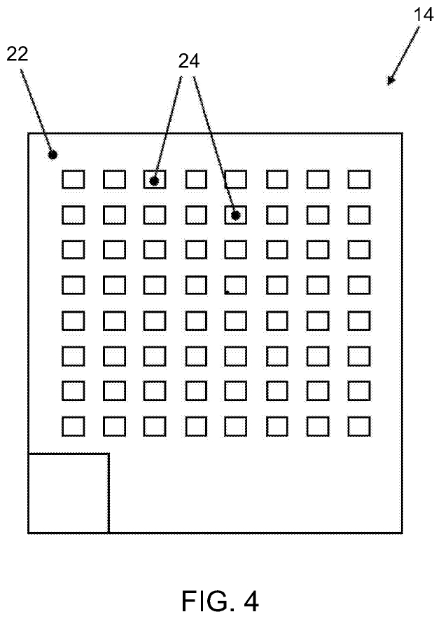

As shown in FIG. 1, the wound dressing 12 comprises a dressing layer 36 with a wound contact surface and a backing layer 38. The electrode component 14 is shown in FIG. 1 between the dressing layer 36 and the backing layer 38 with its apertures (as shown in FIGS. 3 and 4) facing toward the backing layer 38. The insulating layer 22 is shown in FIG. 1 on the uppermost surface of the electrode component 14. The electrode arrangement may be adhered in place, or it may be fixed in place using some suitable mechanical arrangement, or it may be incorporated into the structure of the wound dressing. Other arrangements of the electrode component 14 are possible within the scope of the present invention. For example, electrode component 14 may be located at or integrated with the adhesive layers, facing layer, film layer, located at or integrated within the backing layer 38, or located at or integrated within an absorbent core of the wound dressing 12.

The dressing layer 36 comprises one or more active agents. In the present embodiment these include an anti-microbial or anti-biofilm agent. They may also include a surfactant which could be for example, neutral, anionic, cationic or combinations of these.

As is well known, the technique of anodic stripping voltammetry comprises electrolytic deposition of the relevant metal (silver in the present example) from solution onto the electrode 18, after which the deposited metal is stripped off to produce a signal proportional to the silver concentration in the solution. An alternative to the anodic stripping process is to make continuous measurements. This is appropriate at higher silver concentrations. In either case the required drive signals are applied to the working electrode 14 under control of software/firmware of the microcontroller, and the result is an output signal which provides an indication of metal ion level in the wound dressing 12, which can thus be used to establish whether the dressing remains fit for purpose, or whether it needs to be changed.

Measurements may be taken at chosen time intervals. In the present example the anodic stripping sequence takes about three minutes to complete, and an optional electrode conditioning sequence takes a further 1.5 minutes. So the present embodiment is capable of updating the measure of silver ion concentration at five minute intervals (or continuously, if continuous measurements are being made). The reporting frequency chosen in practice may be lower, however, in order to conserve battery lifetime, e.g. once an hour. The driver electronics 32 buffer the measurement data and upload it to a network interface, which in the present embodiment is in a personal area network (PAN).

The electrode component 14 of the electrochemical sensor 10 needs to be wetted out in order to function. In the present embodiment the dressing layer 36 is moist and the liquid it carries serves to wet out the electrodes. In other embodiments the electrodes may be wholly or partly wetted out by fluid from the wound itself. Wound fluid typically contains 0.9% sodium chloride making it conductive. The electrochemical sensor 10 may incorporate a sensor configured to detect when the system has been bathed in fluid (i.e. when it is operational). This sensor may measure resistance. A reduction in resistance between two electrodes when the wound fluid forms a conductive path between them may be detected as an indication that wound fluid has wetted out the sensor. Multiple electrodes may be provided at spatial intervals across the dressing or through its thickness to establish the degree of spread of exudate.

The wireless interface used to transmit the measurement data to the processing device 13 may take any suitable form. A range of suitable communications protocols is known to the skilled person. In the present embodiment it uses the Bluetooth (RTM) Low Energy standard and the processing device 13 takes the form of a Bluetooth equipped tablet computer or mobile phone running an app for display of the measurement data to the user. A user, which may or may not be a clinician, can thus pair the tablet computer or mobile phone 13 periodically to the electrochemical sensor 10 to obtain the stored data and assess whether the dressing needs to be changed. Alternatively, or additionally the electrochemical sensor 10 may be configured to push a warning signal to the processing device 13 in response to measurement data indicative of a need to change the dressing.

The information provided to the user includes an indication of whether the electrochemical sensor 10 is operational, and can include some form of easily comprehensible indication of the current condition of the wound dressing 12. This may for example be single number, or a colour coded signal, e.g. red for concern, amber for deteriorating condition, and green to indicate good anti-microbial or anti-biofilm level.

The overall configuration of a system embodying the invention is represented in FIG. 2, showing the electrode component 14 whose output is received by the driver electronics 32 and transmitted by network interface 50 of the driver electronics 32 to the processing device 13. The data is additionally or alternatively transmitted, directly by the network interface 50 or via the processing device 13, to a remote host 52. The remote host 52 may collate and analyse data from multiple sources, e.g. in order to facilitate assessment and development of the technology, and to monitor its efficacy.

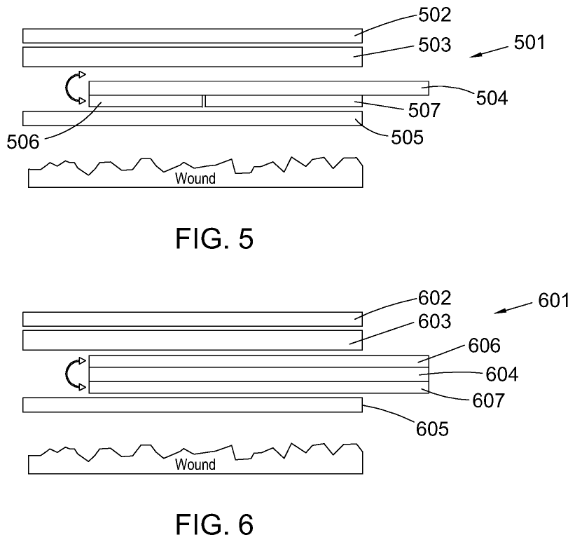

It will be appreciated that the structure of a wound dressing according to the present invention may vary, as required, depending on its use. FIGS. 5-9 illustrate wound dressing structures forming various embodiments of the present invention.

Referring to FIG. 5, there is a depicted a wound dressing 501 according to the present invention configured to monitor concentration of an electroactive species (e.g. anti-microbial or anti-biofilm agent) in or on the wound dressing or in its surrounding environment (e.g skin) while the wound dressing is in use and applied to the body of a patient. The wound dressing 501 has a laminate structure including a backing layer 502, dressing layer 503, a support layer 504 and a wound contact layer 505. The dressing layer 503 and wound contact layer 505 may optionally include an active ingredient (e.g. an anti-microbial or anti-biofilm agent). Fabricated to the wound facing surface of the support layer 504 is an electrode component 506 comprising or consisting of a working electrode configured for sensing electroactive species within the wound environment and a counter (or reference) electrode 507. As depicted by the curved arrow in FIG. 5, it will be appreciated that the electrode component 506 and counter/reference electrode 507 may alternatively be fabricated to the non-wound facing surface of the support later 504.

FIG. 6 also depicts a wound dressing 601 according to the present invention configured to monitor concentration of an electroactive species (e.g. anti-microbial or anti-biofilm agent) in or on the wound dressing or in its surrounding environment while the wound dressing is in use and applied to the body of a patient. Like FIG. 5, FIG. 6 possess a laminate structure including a backing layer 602, dressing layer 603, a support layer 604 and a wound contact layer 605. The dressing layer 603 and wound contact layer 605 may optionally include an active ingredient (e.g. an anti-microbial or anti-biofilm agent). In this embodiment, an electrode component 606 comprising or consisting of a working electrode configured for sensing electroactive species within the wound environment is fabricated to the non-wound facing surface of the support layer 604 whilst the counter/reference electrode 607 is fabricated to the wound facing surface of the support layer 604. Alternatively and illustrated by the curved arrow in FIG. 6 it will be appreciated that electrode component 606 may fabricated to the wound facing surface of the support layer 604 whilst the counter/reference electrode 607 is fabricated to the non-wound facing surface of the support layer 604. Electrode orientations like these, where electrodes are located on opposing surfaces of the support layer 604 can reduce the overall sensor footprint on the wound facing surface on the support layer 604. This, advantageously, decreases the impact that the sensor has on the flow of fluid (e.g. wound exudate) within the wound dressing or in its surrounding environment while the wound dressing is in use and applied to the body of a patient.

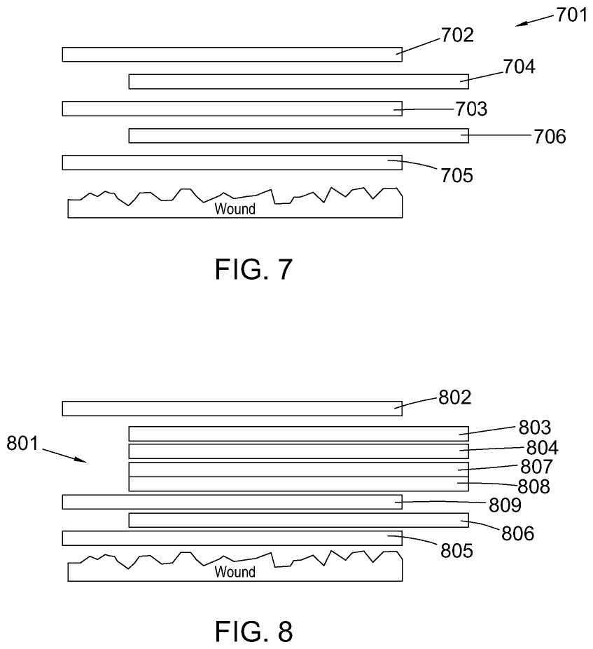

FIG. 7 illustrates a wound dressing 701 according to the present invention configured to monitor concentration of an electroactive species (e.g. anti-microbial or anti-biofilm agent) in or on the wound dressing or in its surrounding environment while the wound dressing is in use and applied to the body of a patient. The wound dressing 701 includes a backing layer 702, counter/reference electrode 704, spacing layer 703, electrode component 706 (comprising or consisting of a working electrode configured for sensing electroactive species within the wound environment wherein the working electrode is in the form of a wire electrode) and wound contact layer 705. As shown in the embodiment of FIG. 7, the electrode component 706 and counter/reference electrode 704 are positioned within the dressing 701 separate from one another and are not fabricated to a common support layer likes FIGS. 5 and 6. The electrode component 706 and counter/reference electrode 704 are configured to be electrically isolated from one another which can be achieved by positioning the electrode on different layers of the dressing structure, or by coating one or both of the electrode with an insulating but porous material. In the embodiment illustrated in FIG. 7, the spacing layer 704 provides an insulating barrier between the electrode component 706 and counter/reference electrode 704 thus electrically isolated them from one another. The wound contact layer 705 may optionally include an active ingredient (e.g. an anti-microbial or anti-biofilm agent). This type of wound dressing arrangement has the advantage that the working wire forming part of the electrode component 706 has minimal impact on fluid flow within the dressing 701. This also reduces the costs associated with the manufacture of the wound dressing since the use of separate electrode components may be less than the cost of manufacturing an integrated sensor.

FIG. 8 depicts a wound dressing 801 according to the present invention configured to release and monitor concentration of an electroactive (e.g. anti-microbial or anti-biofilm agent) in or on the wound or in its surrounding environment while the wound dressing is in use and applied to the body of a patient. The wound dressing includes a backing layer 802, counter/reference electrode 803 and spacing layer 804. The spacing layer 804 electrically isolates the counter/reference electrode 803 from the other conductive components within the wound dressing any may be in the form of an insulating coating formed on the counter/reference electrode 803. The dressing also includes an active release electrode 807, active release layer 808 and another spacing layer 809. The active release electrode 807 and active release layer 808 electrically isolated from other components by virtue of the spacing layer 804 and 809. The purpose of the active release electrode 807 and active release layer 808 is to control release of a species forming part of the active release layer 808 by controlling an electrochemical reaction on the active release electrode which cause release of the electroactive species. The electrode component 806 (comprising or consisting of a working electrode configured for sensing electroactive species within the wound environment wherein) is configured to sense the concentration of the electroactive species being released within the wound dressing environment. Wound dressing 801 also includes a wound contact layer 805.

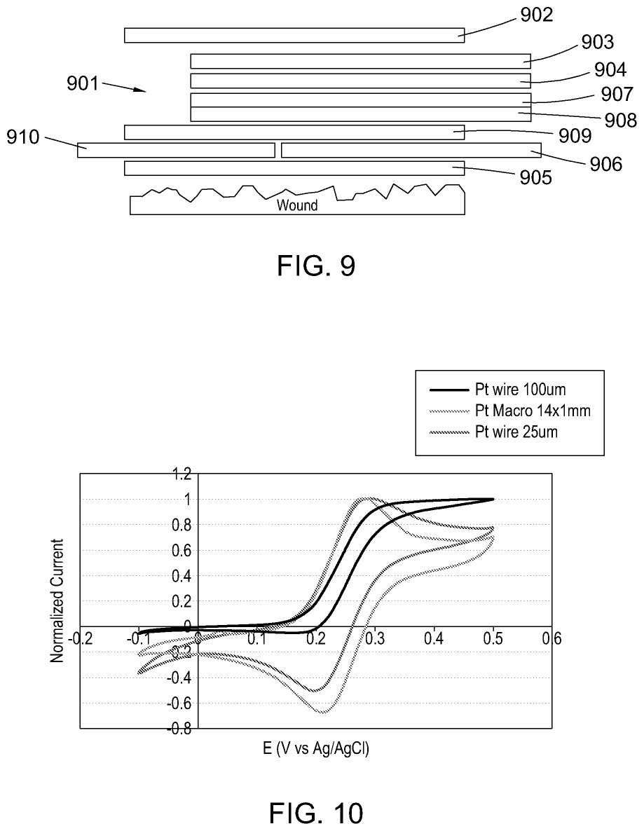

Like FIG. 8, FIG. 9 depicts a wound dressing 901 according to the present invention configured to release and monitor concentration of an electroactive (e.g. anti-microbial or anti-biofilm agent) in or on the wound dressing or in its surrounding environment while the wound dressing is in use and applied to the body of a patient. The wound dressing includes a backing layer 902, counter/reference electrode 903, spacing layer 904, an active release electrode 907, active release layer 908 and another spacing layer 909 as described for wound dressing 801 above. In addition, wound dressing 901 also includes an electrode component 906 (comprising or consisting of a working electrode configured for sensing electroactive species within the wound environment) configured to sense the concentration of the electroactive species being released within the wound dressing environment from the active release electrode 907 and active release layer 908 as described for the embodiment illustrated in FIG. 8. The wound dressing 901 however also includes a wound status sensor 910 configured to monitor a suitable wound status parameter (e.g. temperature, pH, moisture content, toxin levels, signalling compounds, enzyme and/or growth factors being released by the wound). The active release electrode 907, active release layer 908 and wound status sensor 910 are in communication such that an electroactive species can be released from the release layer 908 via an electrochemical reaction on the active release electrode 907 in response to a change in the wound status parameter monitored by wound status electrode 910. The level of the electroactive species is then detected by the electrode component 906.

Wound dressing 901 also includes a wound contact layer 905.

### Experimental Data

### Electrode Types

Tables 1 and 2 describe various parameters for a planar macro electrode, a Nanoband electrode, a number of micro-wire electrodes, a circular planar macro electrode. The Nanoband electrode is flat planar platinum electrode, as depicted in FIGS. 3 and 4. The electrode has a non-conductive substrate layer 20, a dielectric (insulating) layer 22 and a platinum coating 16 (50 nm thickness) on the non-conductive substrate layer, located between the non-conductive substrate layer and the dielectric layer. Square apertures 24 are formed in the electrode by etching down through the dielectric layer and platinum coating to form square apertures having edge dimensions of 30 μm (length)×30 μm (width) with platinum edge (50 nm thickness) exposed as the sensing surface, depicted in FIG. 3 as end faces 18. The apertures have an interval (spacing distance) of 30 μm. This type of Nanoband electrode structure is described in more detail with reference to FIG. 3 and Falk M, Sultana R, Swann M J, Mount A R, Freeman N J. Nanoband array electrode as a platform for high sensitivity enzyme-based glucose biosensing. Bioelectrochemistry. 2016 December; 112:100-5. doi: 10.1016/j.bioelechem.2016.04.002. Epub 2016 Apr. 14. PMID: 27118384.

To provide a meaningful comparison between the electrodes, the dimensions of each electrode (i.e. the electrode length and width show in Table 1) have been chosen such that the surface area of the dressing that is capable of being probed by the electrode, herein referred to as the footprint area probed, is approximately the same for all electrodes, namely approximately 4 mm².

In Tables 1 and 2 below, the “occluded footprint” describes the 2D surface area of the electrode structure. The values calculated for the occluded footprint area are based on the maximum physical dimensions of the electrodes in the plane of the electrode. The footprint area probed is the same, but with the addition of the maximum diffusion length (200 μm) extending beyond the edge of the electrode. When used in a dressing, this surface area corresponds to the area of probe that is adjacent the surface of the wound that may affect or block fluid flowing away from a wound.

For the Nanoband electrodes, the footprint calculations are the same as for the macro electrode. As the array spacing is smaller than the maximum diffusion length, meaning the diffusion fields from adjacent apertures overlap, the volume probed is also approximated to the same as the macro electrode. As the planar electrodes are coated onto a support film this volume only extends on one side of the electrode.

The “solution volume probed” describes the maximum volume of solution that could be probed for a given working electrode during use. It will be appreciated that when the working electrode forms part of a wound dressing the “solution volume probed” is equivalent to the volume of wound fluid (e.g. wound exudate) within the wound environment that could be probed for a given working electrode. The solution volume probed is calculated based on a probing distance of 200 μm which is measured in a perpendicular direction from the surface of the working electrode. In Tables 1 and 2, the solution volume probed is calculated assuming a maximum diffusion length of analyte species to each electrode surface of 200 μm which corresponds to the probing distance mentioned above. Using the Nanoband electrode in Table 2 as an example, the planar dimensions of the electrode are 7 mm×3 mm (equal to the occluded footprint). Assuming a maximum 200 μm diffusion length for an analyte within the solution, this extends the planar dimension of the solution probed to approximately 3.2 mm×7.2 mm. The footprint area probed is therefore 3.2×7.2=23.04 mm². The thickness of solution probed is the diffusion length 200 μm which gives a solution volume probed of 3.2 mm×7.2 mm×0.2 mm=4.6 mm³.

The “exposed surface area of the working electrode” describes the surface area of the working electrode which is exposed to the solution which is being probed and available for sensing (i.e. the area that is capable of receiving ions from the solution). It will be appreciated that when the electrode forms part of a wound dressing the “exposed surface area of the working electrode” is equivalent to the surface area of the working electrode that is exposed to the wound environment during use. That is, the surface area of the working electrode that is available for detecting an electroactive species during use (e.g. the surface area of the electrode which is available for sensing Ag ions within a wound exudate). The “solution volume probed/exposed surface area of the working electrode” describes the maximum volume of solution that the working electrode is capable of probing for a given surface area of exposed working electrode. This provides a guide as to the sensitivity of the working electrode. Taking the example of the embodiment described in FIG. 3, the exposed electrode surface area would correspond to the area of exposed end faces 18.

TABLE 1

| | | | | | | | Solution |
|---|---|---|---|---|---|---|---|
| | | | | | | Exposed | volume probed/ |
| | | | | | | surface | Exposed |
| | Footprint | | | Occluded | Solution | area of | surface area of |
| | area | | | footprint | volume | working | working |
| Electrode | probed | Length | Width | area | probed | electrode | electrode |
| Type | (mm²) | (mm) | (mm) | (mm²) | (mm³) | (mm²) | (mm) |
| Planar Macro | 4.08 | 3.2 | 1 | 3.2 | 0.816 | 3.2 | 0.26 |
| Pt Micro-wire | 4.1 | 8 | 0.1 | 0.8 | 1.541 | 2.513 | 0.61 |
| (100 μm) | | | | | | | |
| Pt Micro-wire | 4.34 | 10 | 0.025 | 0.25 | 1.434 | 0.785 | 1.83 |
| (25 μm) | | | | | | | |
| Pt Micro-wire | 4.13 | 10 | 0.005 | 0.05 | 1.305 | 0.157 | 8.31 |
| (5 μm) | | | | | | | |
| Pt Micro-wire | 4.09 | 10 | 0.001 | 0.01 | 1.280 | 0.031 | 40.74 |
| (1 μm) | | | | | | | |
| Nanoband | 4.08 | 3.2 | 1 | 3.2 | 0.816 | 0.0053 | 153.0 |
| electrode | | | | | | | |
| Circular | 4.52 | 2 | 2 | 3.14 | 0.905 | 3.14 | 0.288 |
| Planar | | | | | | | |
| Macro | | | | | | | |
| (Carbon) | | | | | | | |
| Micro-wire | 4 | 10 | 0.005 | 15 | 0.942 | 0.05 | 18.8 |
| (5 μm, | | | | | | | |
| Carbon) | | | | | | | |

It is desirable for the solution volume probed/exposed surface area of working electrode ratio to be as high as possible. This is attributed to a high degree of sensitivity (signal to noise) and reduced tendency for the signal to deteriorate due to mass transport hindrances.

As can be seen in Table 1, micro-wires have the smallest occluded footprint and therefore have the smallest impact on the flow of fluid away from the wound and into the dressing. The solution volume probed is also increased when using wire electrodes over planar electrodes. Without wishing to be bound by theory, this is because diffusion is available from all sides of the wire (i.e. from an area radially surrounding the wire).

However, the solution volume probed per electrode area, and therefore the sensitivity (i.e. signal to noise) of the working electrode, is greatest for the planar Nanoband electrode. This arises due to the highly reduced exposed surface area of the Nanoband electrode.

The experiments detailed below were performed using the electrodes detailed in Table 2. The structure of the planar macro electrode is as described above for Table 1. The Nanoband electrode structure is as described above and includes edge dimensions of 30 μm (length)×30 μm (width) with an exposed platinum edge thickness of 50 nm and aperture spacing intervals of 30 μm. The Nanoband electrode includes approximately 5830 apertures.

Table 2 shows the dimensions of the electrodes used in the accompanying experimental studies described below.

TABLE 2

| | | | | | | | Solution |
|---|---|---|---|---|---|---|---|
| | | | | | | Exposed | volume probed/ |
| | | | | | | surface | Exposed |
| | Footprint | | | Occluded | Solution | area of | surface area of |
| | area | | | footprint | volume | working | working |
| Electrode | probed | Length | Width | area | probed | electrode | electrode |
| Type | (mm²) | (mm) | (mm) | (mm²) | (mm³) | (mm²) | (mm) |
| Planar | 8.6 | 7 | 1 | 7 | 1.728 | 7 | 0.247 |
| Macro (Pt) | | | | | | | |
| Micro-wire | 12.6 | 25 | 0.1 | 2.5 | 4.745 | 7.85 | 0.604 |
| (100 μm, | | | | | | | |
| Pt) | | | | | | | |
| Micro-wire | 10.7 | 25 | 0.025 | 0.625 | 3.554 | 1.96 | 1.81 |
| (25 μm, Pt) | | | | | | | |
| Nanoband | 23.0 | 7 | 3 | 21 | 4.608 | 0.035 | 131.7 |
| electrode | | | | | | | |

Mass transport to micro electrodes vs mass transport to macro electrodes FIG. 10 shows the results of three cyclic voltammetry experiments, where the potential (E) of different electrodes were swept between −0.1 and 0.5 V with respect to a Ag/AgCl electrode in 1 mM ferrocene carboxylic acid prepared in phosphate buffered saline (PBS) solution and platinum counter electrode. The planar electrodes were coated onto a polyethylene terephthalate (PET) film. The electrodes assessed as shown in FIG. 10 were a macro platinum (Pt) planar electrode 14 mm×1 mm (shown as green), a 100 μm platinum wire electrode (shown as blue) and a 25 μm platinum wire electrode (shown in black) (as described in Table 2 above). The data for the 25 μm platinum wire electrode shows a classic sigmoidal shape with no drop in current above a potential of 0.3V. The 100 μm platinum wire electrode shows some drop in current above 0.3V and the macro electrode shows the largest drop in current at this potential. Without wishing to be bound by theory, this effect occurs due to a mass transport limitation. Importantly, it has been observed that for measuring low concentrations of Ag ions using macro electrodes in the system illustrated in FIG. 10, it is usually necessary to rotate the electrode rapidly to provide sufficient mass transport to the electrode surface. This requirement to agitate the electrode is of course impractical when the electrode is intended to be housed within a wound dressing. Advantageously, this requirement is eliminated when using micro or nano electrodes according to the invention, rendering the working electrodes of the present invention eminently suitable for use in wound care applications.

### Use of Anodic Stripping Voltammetry to Measure Silver Ions

To detect low concentration of metal ions, such as silver, electrochemical methods such as anodic stripping voltammetry can be used. Anodic stripping voltammetry reduces the metal ions onto the electrode surface over a certain period of time and then re-oxidises them in one step to produce a clearly measurable signal.

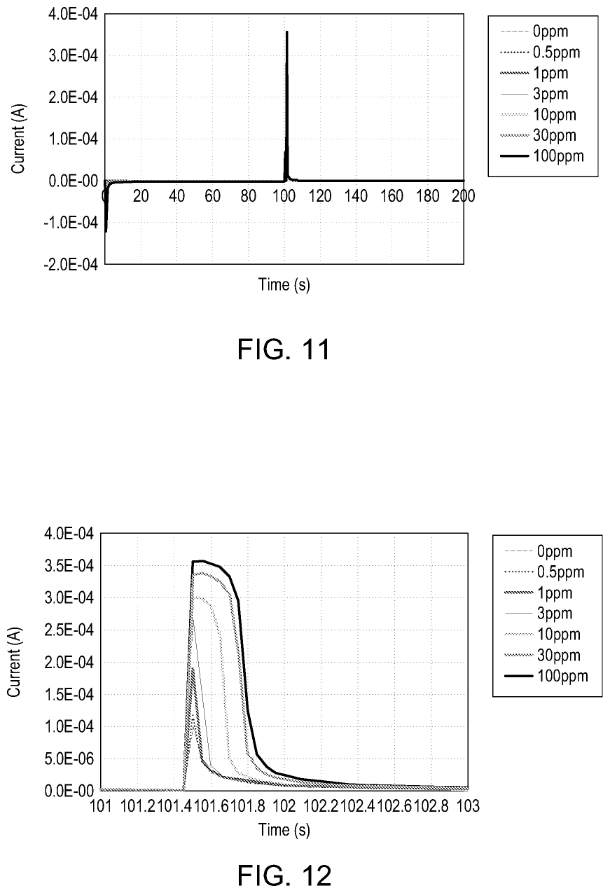

FIG. 11 shows the measured current over time from seven anodic stripping voltammetry experiments conducted in Mueller-Hinton Broth II (MHB2) solutions that contained differing concentrations of added Ag⁺ ions. The concentrations ranged between 0 ppm and 100 ppm and the electrode was a platinum Nanoband electrode supplied by NanoFlex Ltd as described above in connection with Table 2. In each experiment, the potential of the electrode was initially held at −0.2V with respect to a standard/reference Ag/AgCl electrode for 100 seconds. The potential was then increased to −0.05V and held for 1 second, and then further increased to +0.2V and held for another 100 seconds. The initial 100 s is the reductive collection stage and the second 100 s is the anodic re-oxidation stage.

FIG. 12 shows an expanded section of FIG. 11 that focuses on the oxidation current observed for each concentration of Ag⁺ ions during the beginning of the anodic re-oxidation stage (the stripping step). The concentration values given in FIGS. 11 and 12 are nominal values.

The data shown in FIGS. 11 and 12 can be used calculate the oxidation charge during the anodic stripping step as a function of the Ag⁺ ion concentration added to the MHB2.

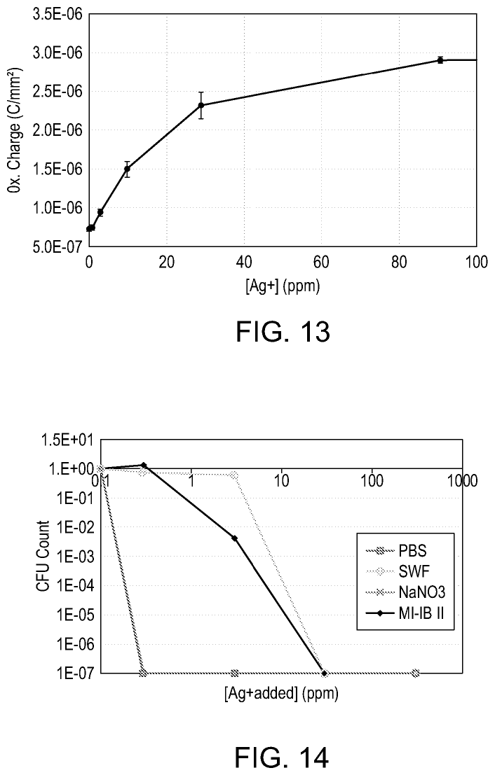

This is displayed in FIG. 13 plotted using the precise values for concentrations instead of the nominal concentrations. The results show that a significant response is observed for concentrations above 1 ppm. Without wishing to be bound by theory, the slope is not linear because at higher concentrations AgCl is precipitated from the solution. The response observed in FIG. 13 is surprising, as the amount of chloride in the solution should limit the Ag⁺ concentration above −1 ppm. MHB2 Contains 17.5 g/L Casein hydrolysate and 3 g/L beef extract and so is unlikely to contain the higher affinity metal ion binding sites that are found in serum albumins, but may contribute to stabilizing Ag⁺ or nanoscale Ag/CI aggregates in solution.

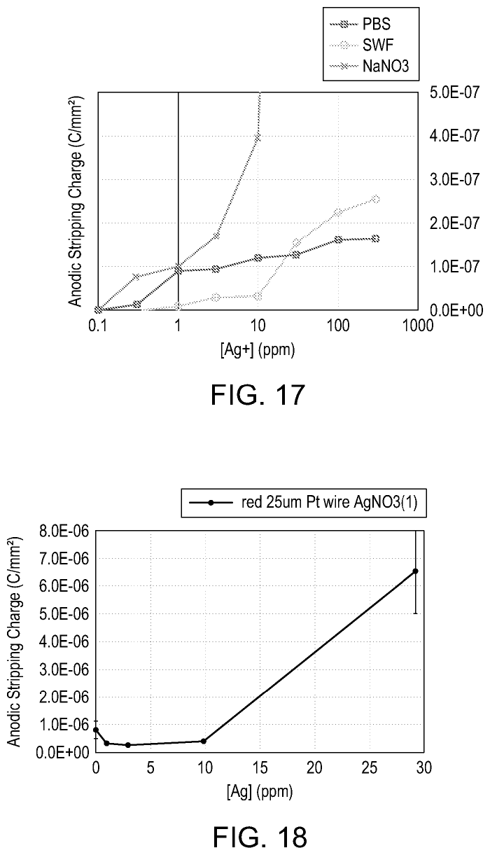

FIG. 16 plots the oxidation charge against Ag⁺ ion concentration obtained from similar anodic striping experiments using the Pt Nanoband electrode but in PBS, Silver Nitrate (NaNO3) and simulated wound fluid (SWF) solutions. Simulated wound fluid was made up of 33 g/L BSA, 100 mM NaCl, 40 mM NaHCO₃, 4 mM KCl and 2.5 mM CaCl₂. (see Bradford C, Freeman R, Percival SL. In vitro study of sustained anti-microbial activity of a new silver alginate dressing. J Am Col Certif Wound Spec. 2009; 1(4):117-120. Published 2009 Oct. 6. doi:10.1016/j.jcws.2009.09.001). In these experiments, Ag⁺ ions were added at concentrations between 0 ppm and 300 μm. The data in FIG. 16 is presented after a blank background subtraction. FIG. 17 plots the same data as FIG. 16 but on a log scale. The data for the simulated wound fluid in FIGS. 16 and 17 are particularly relevant, showing a strong response at and above 30 ppm.

Similar anodic striping experiments were also conducted using microelectrodes. FIG. 18 displays the oxidation charge as a function of Ag⁺ concentration using a 25 μm Pt wire electrode. The Ag⁺ ions were added to a simulated wound fluid (SWF) containing 33 g/L bovine serum albumin (BSA). Each point displayed is the average of a duplicate sequential measurements, and associated error bars have been calculated. As seen for the Pt nanoband electrode, the sensor response is evident at 30 ppm and above. Without wishing to be bound by theory, this may be because the serum albumin, which contains higher affinity metal ion binding sites reduces the effective Ag⁺ concentration until the added Ag⁺ concentration approaches that of the BSA. The molar concentration of 33 g/L BSA is 0.5 mM and 30 ppm Ag⁺ corresponds to 0.28 mM.

### Bacterial Proliferation Assays

To assess the antibacterial efficacy of the silver, and to correlate this with the sensor response, bacterial proliferation assays were performed on MHB2, PBS, NaNO3 and SWF solutions with added silver at concentrations between 0.3 to 300 ppm. FIG. 14 shows the number of Colony Forming Units of Pseudomonas that were measured after a bacterial proliferation assay in the different solutions as a function of the amount of added Ag⁺.

The bacterial proliferation assay data shows that in NaNO₃ and PBS solutions the efficacy of the Ag⁺ is already apparent at the lowest concentration measured (0.3 ppm), however in SWF solutions the efficacy of Ag⁺ is seen only for 30 ppm added Ag⁺ and above. This mirrors the electrochemical sensor data which shows a poor response of the sensor to Ag⁺ below 30 ppm, presumably due to preferential binding to the BSA in SWF.

### Optical Density Measurements

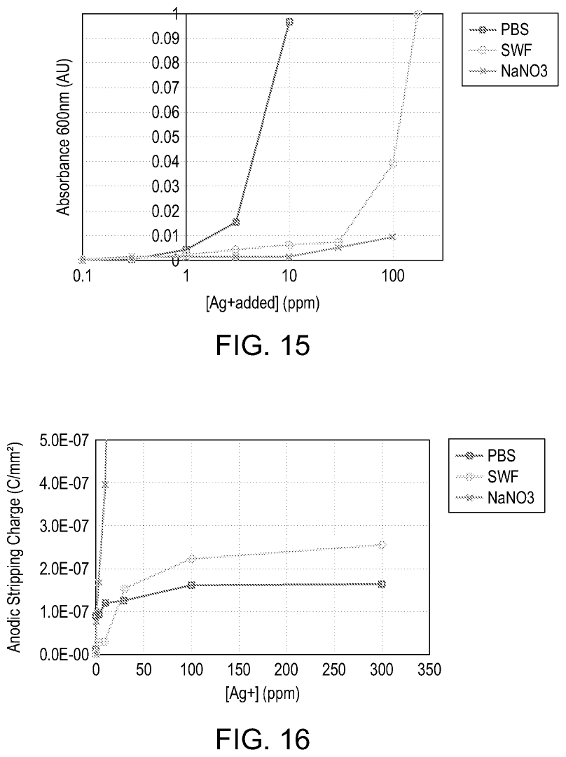

Optical density measurements can be used to indicate the presence of AgCl precipitates/aggregates in solution. FIG. 15 shows Optical density data measurements for solutions of PBS, SWF and NaNO₃ as a function of concentration of added Ag⁺ ions. No bacteria where present for the measurements. This OD data shows the impact of AgCl precipitation in SWF above 30 ppm and in PBS above 1 ppm, which result in the nonlinear sensor measurements.

Using Multiple Anodic steps

The effect of the anodic stripping technique is to concentrate the reduced metal ion (Ag) onto the sensing electrode and to re-oxidise it in an anodic step. Due to the poor solubility of Ag⁺ in SWF and other chloride containing media, this process, which generates high concentrations of Ag⁺ at the electrode surface, can have the effect of precipitating insoluble silver salts. These salts are then present and can be re-reduced at the next measurement increasing the subsequent anodic stripping signal and enhancing the size of the silver signal. When silver levels in the SWF reduce again, this additional silver is solubilized and the signal reduces. This effect can be ameliorated by using a short initial reduction step and progressively increasing the reduction time until a signal is detected, which limits the amount of Ag deposited on the electrode. Further, instead of a single anodic stripping potential step, either a slow voltage scan or multiple smaller potential steps can be used, such that the silver ions are released at a reduced rate back into the solution. This is relevant for high concentrations of Ag⁺. Another advantage of performing multiple small potential steps is that once above the maximum silver oxidation potential, the charge passed can be used as a measure of the size of the background charging current present during the Ag oxidation steps.

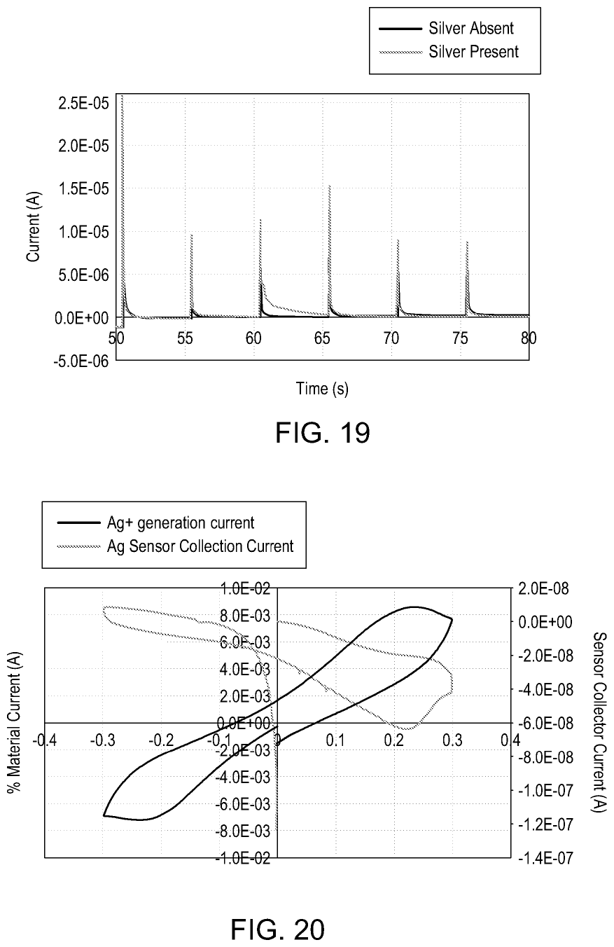

FIG. 19 shows the current passed during multiple anodic steps for two nanoband electrodes after 50 s at a reducing potential (−0.2V) in simulated wound fluid, one in the presence of added silver (30 ppm) and one without any added silver. The steps observed in the current after 55 s correspond to incremental increases of the potential by 0.05V, starting at −0.05V. The step at 60 s shows anodic stripping of the deposited Ag, whereas the steps before and after are just the background charging capacitance of the electrode.

### Electrochemical Ag⁺ Generation

FIG. 20 shows the results of an experiment designed to generate and detect Ag⁺ ions in a gauze dressing. The experiment involved a Ag/AgCl counter/reference electrode, a Ag/AgCl silver release electrode and a Pt nanoband sensor (as described in Table 2). During the experiment, the potential of the Ag/AgCl release electrode (as shown in blue) is scanned (0V to −0.3V to +0.3V to 0V) to reduce and then oxidize silver. Meanwhile the Ag sensing electrode (orange) is held at −0.2V. At this potential Ag⁺ ions in solution can be reduced on the sensing electrode surface producing a negative sensor collector current. The sensor collector current is shown on the right hand Y-axis, while the current of the release electrode is shown on the central axis. Note the two axes do not share the same origin. The x-axis on the plot meanwhile shows the potential of the Ag release electrode being scanned. Initially when the experiment starts at 0V and scans to negative potentials, there is a large reduction in the magnitude of the negative sensor collector current on the sensing electrode. This current reaches zero as the Ag electrode scans to −0.3V to reduce silver. As the scan then increases the potential of the release electrode and goes above zero, the Ag release electrode passes positive current, oxidizing Ag to Ag⁺ which is in part released into the solution. The magnitude of the negative reduction current on the Ag sensing electrode therefore increases again as it reduces the Ag⁺ ions in solution that were produced by the generator (the Ag release electrode). Finally, as the release electrode potential is reduced back to zero and less silver is generated, the reduction current of the sensor electrode also returns to zero.

### Detection of Non-Metallic Anti-Microbial/Anti-Biofilm Agent (Iodine)

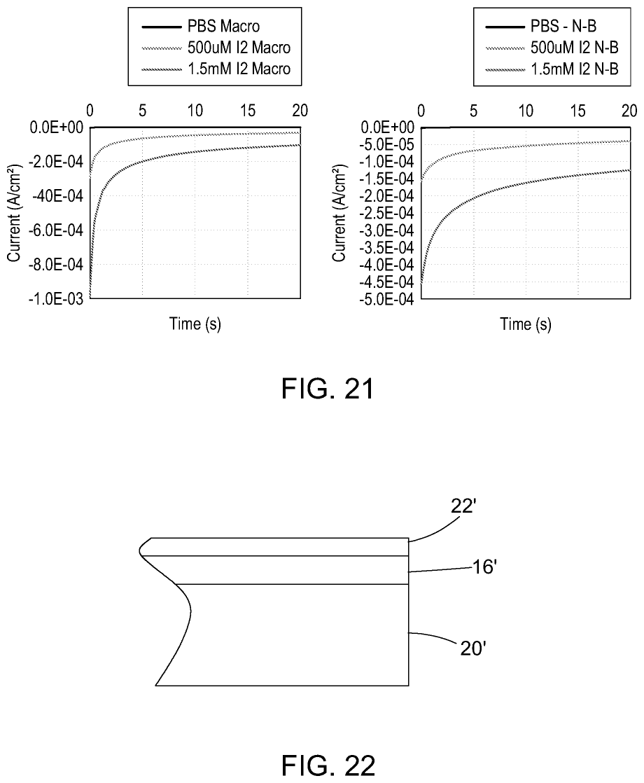

In this example, the measurement of a non-metallic anti-microbial/anti-biofilm agent using a nano-electrode is performed. The data shown in FIG. 21 illustrates the measurement of iodine in phosphate buffered saline (PBS). Iodine can be measured either at oxidative or at reductive potentials. The plot on the left hand side of FIG. 21 shows the measurement of iodine using a platinum based macro electrode (as described in Table 1 above under “Planar Macro”) at iodine concentrations of 500 μM and 1.5 mM.

The plot on the right hand side of FIG. 21 shows the measurement of iodine using a Pt nanoband array sensor with printed Ag/AgCl reference electrode and Platinum counter electrode (as described in Table 1 above under “Nanoband electrode”) at iodine concentrations of 500 μM and 1.5 mM The data in left hand side plot of FIG. 21 shows the reduction of iodine as the curve moves towards a potential of 0V (vs. Ag/AgCl) as function of time. The response shown in the right hand side plot for the nanoband electrode shows significantly less decay with time due to improved diffusion and analyte depletion characteristics as compared to the macro electrode. This results in a more stable measurement being achieved by the nanoband electrode. This example demonstrates that the use of micro or nano electrodes over an extended area of a wound dressing for the measurement of anti-microbial/anti-biofilm agents will result in less depletion of the anti-microbial/anti-biofilm agent levels as a result of the measurement process itself. This overcomes the analyte depletion issues associated with macro electrodes where agent is significantly consumed as part of the measurement mechanism which can lead to an unstable analyte measurement.

FIG. 22 is a schematic representation of a micro-wire or micro-strip electrochemical sensor arrangement according to the present invention. The schematic shows a cross section of the arrangement with the electrode labelled as 16′, the insulating layer labelled as 22′ and substrate layer labelled as 20′.

### Cyclic Voltammetry Experiments Conducted Using a Carbon Micro-Wire Electrode

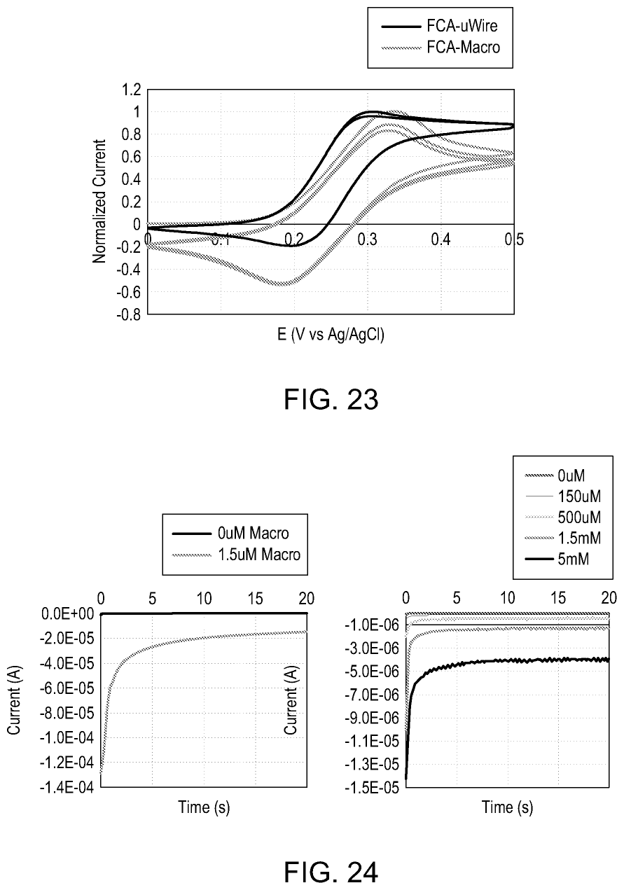

FIG. 23 shows a normalised cyclic voltammogram of a carbon micro-wire electrode in 1 mM Ferrocene Carboxylic Acid in PBS. The plot displays a sigmoidal shape which is characteristic of micro-electrodes and in contrast to a macro electrode also plotted. The current is normalised to the maximum current for each electrode. The macro electrode is a 3.3 mm diameter circular printed carbon electrode on a PET substrate, which is part of a 3-electrode electrochemical sensor with carbon counter electrode and Ag/AgCl reference electrode. The circular surface and edges of the marco carbon electrode are exposed for analyte detection. The micro-wire electrode in this example has a width (or thickness) of approximately 5 μm and a length of approximately 3.3 mm.

FIG. 24 shows the measurement of iodine (at varying concentrations) with a carbon micro-wire electrode as compared to a conventional macroscopic printed carbon electrode. Both are utilised with a Ag/AgCl reference electrode and carbon counter electrodes. The macro electrode is a 3.3 mm diameter circular printed carbon electrode on a PET substrate, which is part of a 3-electrode electrochemical sensor with carbon counter electrode and Ag/AgCl reference electrode. The circular surface and edges of the marco carbon electrode are exposed for analyte detection. The micro-wire electrode in this example has a width (or thickness) of approximately 5 μm and a length of approximately 3.3 mm. As can be seen from the plot on the right hand side, the micro-wire sensor rapidly reaches a steady state measurement.

The dimensions of the carbon macro electrode and carbon micro-wire utilisted for the experiments illustrated in FIGS. 23 and 24 are provided in Table 3 below.

TABLE 3

| | | | | | | | Solution |
|---|---|---|---|---|---|---|---|
| | | | | | | Exposed | volume probed/ |
| | | | | | | surface | Exposed |
| | Footprint | | | Occluded | Solution | area of | surface area of |
| | area | | | footprint | volume | working | working |
| Electrode | probed | Length | Width | area | probed | electrode | electrode |
| Type | (mm²) | (mm) | (mm) | (mm²) | (mm³) | (mm²) | (mm) |
| Circular | 10.75 | 3.3 | 3.3 | 8.55 | 2.15 | 8.553 | 0.251 |
| Planar | | | | | | | |
| Macro (C) | | | | | | | |
| Micro-wire | 1.32 | 3.3 | 0.005 | 4.95 | 0.311 | 0.0165 | 18.8 |
| (5 μm, C) | | | | | | | |

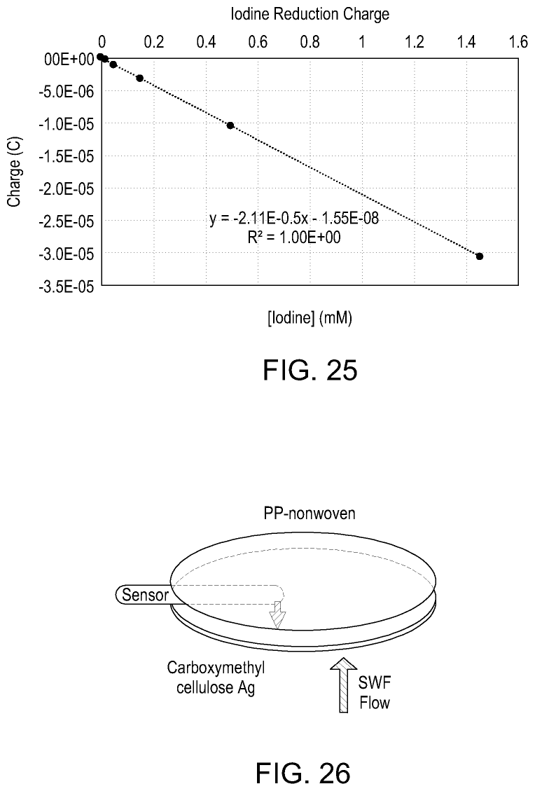

FIG. 25 shows a graph plotting the iodine reduction charge versus concentration for a printed carbon micro-wire electrode having a width (or thickness) of approximately 5 μm and a length of approximately 3.3 mm.

### Simulated Wound Fluid Flow Test

FIG. 26 shows a schematic of a nanoband electrode sensor (as described in Table 1 above) incorporated within a wound dressing arrangement. The wound dressing arrangement includes a silver loaded carboxymethyl cellulose dressing material as a lower layer and a polypropylene (nonwoven) upper layer. The sensor component is sandwiched between the carboxymethyl cellulose dressing layer and polypropylene layer. The sensor dressing is mounted into a flow cell for measurement under simulated wound fluid (SWF). The blue arrows represents the direction of flow of the simulated wound fluid whereas the red arrow represents the direction that the working electrode is facing in this setup.

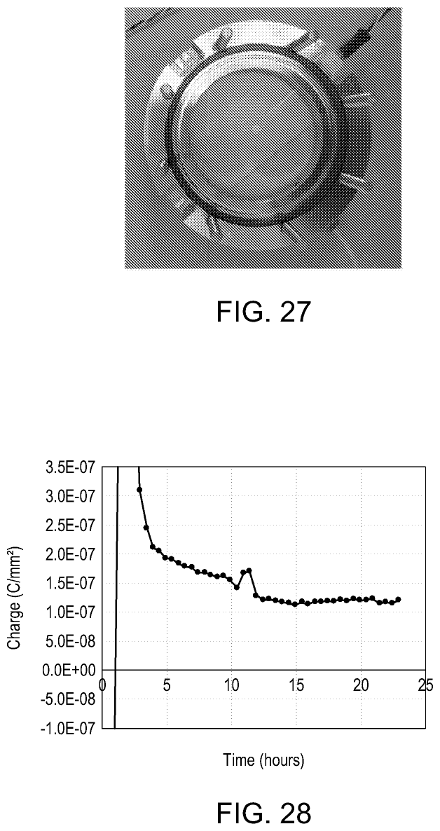

FIG. 27 provides a photograph of the type of wound dressing sensor represented in FIG. 26 mounted within a flow cell.

FIG. 28 shows the results from an experiment performed with a nanoband electrode sensor (as described in Table 1 above) incorporated within a wound dressing arrangement as shown in FIGS. 26-27 which is subjected to flow of simulated wound fluid over the course of 24 hours at a flow rate of 2 mL/hour. The decaying signal for silver, measured by the sensor, can be clearly seen as time progresses and can be used as an indication for when the levels of active silver within the wound fluid drop below therapeutic levels and the wound dressing needs to be changed.

It will be appreciated that numerous modifications to the above described sensor may be made without departing from the spirit and scope of the invention, for instance, the scope of the invention as defined in the appended claims. Moreover, any one or more of the above aspects/embodiments could be combined with one or more feature of the other aspects/embodiments and all such combinations are intended with the present disclosure.

Optional and/or preferred features may be used in other combinations beyond those explicitly described herein and optional and/or preferred features described in relation to one aspect of the invention may also be present in another aspect of the invention, where appropriate. The described and illustrated embodiments are to be considered as illustrative and not restrictive in character, it being understood that only the preferred embodiments have been shown and described and that all change and modifications that come within the scope of the invention as defined in the claims are desired to be protected.

It should be understood that while the use of words such as “preferable”, “preferably”, “preferred”, or “more preferred” in the description suggest that a feature so described may be desirable, it may nevertheless not be necessary and embodiments lacking such a feature may be contemplated as within the scope of the invention as defined in the appended claims. In relation to the claims, it is intended that when words such as “a”, “an” or “at least one” are used to preface a feature there is no intention to limit the claim to only one such feature unless specifically stated to the contrary.

1. A wound dressing comprising:
an electrochemical sensor configured to detect the level of an electroactive species within a wound environment during use, the electrochemical sensor comprising a working electrode; and
wherein the working electrode is a micro electrode or nano electrode.

2. The wound dressing of claim 1, wherein the electrochemical sensor detects the electroactive species by anodic stripping voltammetry, amperometry, potentiometry or electrochemical impedance analysis, preferably, wherein the electrochemical sensor detects the electroactive species by anodic stripping voltammetry.

3. The wound dressing of claim 1, wherein the electroactive species is an anti-microbial agent and/or anti-biofilm agent.

4. The wound dressing of claim 1, wherein:
(i) the electroactive species is a metal ion selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr ions; or
(ii) the electroactive species is a non-metal species.

5. The wound dressing of claim 1, wherein the surface area of the working electrode that is exposed to the wound environment during use (exposed surface area of the working electrode) is from about 0.0005 mm² to about 10 mm².

6. The wound dressing of claim 1, wherein (solution volume probed)/(exposed surface area of the working electrode) is from about 0.5 mm to about 300 mm.

7. The wound dressing of claim 1, wherein the working electrode is elongate, optionally wherein the working electrode is a wire.

8. The wound dressing of claim 7, wherein the wire has a thickness of from about 0.1 μm to about 150 μm.

9. The wound dressing of claim 7, wherein (solution volume probed)/(exposed surface area of the working electrode) is from about 0.5 mm to about 100 mm.

10. The wound dressing of claim 1, wherein the working electrode is planar.

11. The wound dressing of claim 10, wherein:
the working electrode is a planar laminate structure comprising a working electrode layer, insulating layer and substrate layer, wherein the working electrode layer is sandwiched between the substrate layer and insulating layer to form the laminate structure;
the laminate structure includes at least one aperture extending through the insulating layer and the working electrode layer to leave a surface of the working electrode layer exposed to the wound environment during use in order to provide a sensing surface suitable for detecting the electroactive species within the wound environment; and
the surface of the working electrode layer exposed comprises exposed edges which are formed at the periphery of the at least one aperture and the exposed edges have a thickness of from about 25 nm to about 75 nm.

12. The wound dressing of claim 10, wherein (solution volume probed)/(exposed surface area of the working electrode) is from about 100 mm to about 200 mm.

13. The wound dressing of claim 1, further comprising a counter and/or reference electrode.

14. The wound dressing of claim 1, further comprising a support layer for the working electrode.

15. A method of detecting the level of an electroactive species within a wound environment comprising:
applying the wound dressing of claim 1 onto a wound; and
operating the electrochemical sensor to detect the electroactive species within the wound environment.

16. The method of claim 15, wherein the operating the electrochemical sensor comprises detecting the electroactive species by anodic stripping voltammetry, amperometry, potentiometry or electrochemical impedance analysis, preferably by anodic stripping voltammetry.

17. The method of claim 15, further comprising administering an anti-microbial agent and/or anti-biofilm agent into the wound in response to the level of electroactive species detected in the wound environment; optionally wherein the electroactive species detected is identical to the administered anti-microbial agent and/or anti-biofilm agent.

18. The method of claim 15, wherein:
(i) the electroactive species is a metal ion selected from the group consisting of Ag, Al, Au, Ba, Bi, TI, Ce, Co, Cr, Cu, Fe, Ga, Ge, Ir, Mn, Mo, Ni, Pd, Pt, Rb, Re, Rh, Ru, Sb, Se, Sn, Sr, Ti, W, Zn and Zr ions; or
(ii) the electroactive species is a non-metal species.

19. (canceled)
